(19) United States
(12) Patent Application Publication
(10) Pub. No.: US 2016/0074234 A1
(43) Pub. Date: Mar. 17, 2016

(54) RADIAL COMPRESSION UTILIZING A SHAPE-MEMORY ALLOY

(71) Applicant: DREXEL UNIVERSITY, Philadelphia, PA (US)

(72) Inventors: Pramod ABICHANDANI, Philadelphia, PA (US); Eric DYKE, Huntingdon Valley, PA (US); William MCINTYRE, Broomall, PA (US); David WYKES, Royersford, PA (US)

(73) Assignee: Drexel University, Philadelphia, PA (US)

(21) Appl. No.: 14/784,296

(22) PCT Filed: Apr. 14, 2014
(86) PCT No.: PCT/US2014/033940

§ 371 Date: Oct. 14, 2015

Related U.S. Application Data
(60) Provisional application No. 61/812,399, filed on Apr. 16, 2013.
(60) Provisional application No. 61/880,342, filed on Sep. 20, 2013.

(51) Int. Cl. A61F 13/08; A61H 1/00; G05B 15/02; A61F 13/00

(52) U.S. Cl. CPC A61F 13/08; A61F 13/00038; A61H 1/008; G05B 15/02; A61F 2013/0028; A61H 2201/165; A61H 2209/00

(57) ABSTRACT

Radial compression may utilize a shape memory alloy. The shape-memory alloy may comprise nickel titanium. A compressive force may be applied to a body part of an animal. For example, the device may be utilized to provide a compressive force to a limb of a human. The device may be utilized to provide compressive therapy to treat patients that suffer from, for example, chronic venous insufficiency or neuromuscular disorders, for recreational massage, or the like. Wires comprising a shape-memory alloy may be wound around an object. The wires may be individually, electrically controlled to provided radial compression. Radial compression utilizing a shape memory alloy concurrently may provide compressive force and thermal energy to an object.

### CROSS REFERENCE TO RELATED APPLICATIONS

The instant application claims the benefit of U.S. provisional patent application No. 61/812,399, filed Apr. 16, 2013. The instant application also claims the benefit of U.S. provisional patent application No. 61/880,342, filed Sep. 20, 2013. U.S. provisional patent application No. 61/812,399 is incorporated by reference herein in its entirety. U.S. provisional patent application No. 61/880,342 is incorporated by reference herein in its entirety.

### BACKGROUND

Many people may exhibit conditions such as chronic venous insufficiency (CVI) and/or lymphedema. Persons with CVI may suffer from malfunctioning venous valves, and thus, poor blood flow back towards the heart. Symptoms may include skin discoloration, dermatitis, venous ulcers, hypoxia, or the like. In some cases, CVI may lead to secondary lymphedema. Secondary lymphedema may result from a disruption of the lymphatic system (e.g., lymph nodes have been damaged or surgically removed). As lymphedema progresses, the affected area may begin to swell as bodily fluids cannot be drained away. In extreme cases, the inflicted area may require reconstructive surgery to reduce swelling and remove damaged tissue.

### SUMMARY

Compression therapy may utilize a shape-memory allow (SMA). Compressive therapy may be utilized to manage, for example, symptoms resulting from CVI and/or lymphedema. Compressive therapy may be utilized to treat patients that suffer from, for example, chronic venous insufficiency or neuromuscular disorders. Compression therapy may include applying compressive force to a body part of an animal, such as, for example, a limb of a human.

Compression therapy utilizing a shape-memory-alloy (SMA) may offer innovative methods of actuation via their shape-memory response to active heating. A device for providing compressive force may comprise a shape-memory allow. The shape-memory alloy may comprise nickel titanium. The shape-memory alloy may comprise an SMA wire. In an example embodiment, the shape-memory alloy may comprise FLEXINOL® wires.

As described herein, radial forces may be generated by subjecting SMA wires to radial actuation. The radial forces may be experienced in applications where SMA wires are wound around an object to provide compressive forces, e.g., compression therapy for patients that suffer from chronic venous insufficiency (CVI). In an example embodiment, an SMA wire may be wound around a cylindrical object, a body part, or the like, and powered using an adjustable constant power supply. Force-sensitive impedance may be utilized to measure the resulting distributed radial force on the object, body part, or the like. The wire's impedance during radial compression also may be measured. The local temperature immediately next to the SMA wire may be recorded. Radial force, SMA wire impedance, and local temperature may be analyzed to assess performance and/or to adjust performance.

As described herein, test results indicate that the maximum distributed radial force may exceed 3.75 F_{kg}. Furthermore, for particular tests reaching the maximum observed force, nearly half of the generated force may occur while the SMA wire is in the austenite phase. Further analysis reveals a linear relationship between input power and maximum generated force for a given power-on period. Trends such as transformation time and rate change of resistance also are described herein. Additionally, force ranges may vary dependent upon SMA wire diameter.

### BRIEF DESCRIPTION OF THE DRAWINGS

The systems, methods, and computer readable media for implementing radial compression using a shape-memory allow are further described with reference to the accompanying drawings in which:

FIG. 1 depicts an example radial compression rig.

FIG. 2 depicts an example instrumentation system.

FIG. 3 depicts an example graphic illustration of radial force versus time for five power cycles.

FIG. 4 depicts an example graphic illustration of resistance versus time for five power cycles.

FIG. 5 depicts an example graphic illustration of resistance versus time for a single power cycle.

FIG. 6 depicts an example graphic illustration of the effect of varying input power level on local temperature.

FIG. 7 depicts an example graphic illustration of resistance versus force.

FIG. 8 depicts an example graphic illustration of phase transformation time versus input power.

FIG. 9 depicts an example graphic illustration of the rate of change of wire resistance for different input power levels.

FIG. 10 depicts another example graphic illustration of the rate of change of wire resistance for different input power levels.

FIG. 11 depicts an example graphic illustration of maximum attainable force versus input power for different power-on times.

FIG. 12 is an example graph depicting resistance vs. force for five power cycles.

FIG. 13 is an example graph depicting the rate of change of resistance with respect to time under different power settings.

FIG. 14 is another example graph depicting the rate of change of resistance with respect to time under different power settings.

FIG. 15 is an example graph depicting the maximum force vs. power for different power-on times.

FIG. 16 depicts an example prototype sleeve.

FIG. 17 is another example instrumentation system.

FIG. 18 is an example schematic diagram of the power regulation system and sensory equipment.

FIG. 19 shows a portion of the SMA sleeve illustrating the sewn restraining tunnels and the zig-zag path of thread sewn into the fabric used to align the SMA wires to the center of each restraining tunnel.

FIG. 20 shows the exposed rubber strips inside the sleeve before they were cut to length and the sleeve was sewn shut.

FIG. 21 shows an example internal electrical terminal with SMA wires and common return wire connected.

FIG. 22 shows an example external terminal and power connection wires.

FIG. 23 shows terminals sewn into the sleeve.

FIG. 24 shows an example rig comprising a polyvinyl chloride (PVC) base.

FIG. 25 is another depiction of the example rig comprising the PVC base.

FIG. 26 depicts an example user interface for controlling of the massaging sleeve.

FIG. 27 is an example graph of force generated over time with respect to input power.

FIG. 28 is a flow diagram of an example process for controlling an SMA compression sleeve.

FIG. 29 is an example block diagram of a system for controlling an SMA compression sleeve.

FIG. 30 shows a table of example user-available ranges of operation of the massaging sleeve.

FIG. 31 illustrates example graphs of normalized resistance versus time for a two second activation period, at 2.2 W/Ω over three actuation periods.

FIG. 32 illustrates example graphs of force versus normalized resistance.

FIG. 33 is an example graph depicting force vs. time wherein force was maintained over a six-second period when compressing a deformable object.

FIG. 34 is an example graph depicting resistance versus time.

FIG. 35 is an example graph depicting force versus time for five power cycles.

FIG. 36 is an example graph of resistance of the SMA wire vs. radial force exerted by the SMA wire.

FIG. 37 is an example plot depicting transformation (transition) times vs. power.

FIG. 38 is an example plot depicting maximum force vs. power for different power-on times.

### DETAILED DESCRIPTION OF ILLUSTRATIVE EMBODIMENTS

Shape-memory-alloys (SMAs) offer innovative methods of actuation via their shape-memory response to active heating. When an SMA is actively heated using heat conduction or Joule heating, a phase transformation may cause the wire to forcefully return to its memorized (annealed) shape. As described herein, the use of SMAs for radial compression therapy may facilitate management of conditions such as chronic venous insufficiency (CVI), lymphedema, or the like. As described above, persons with CVI suffer from malfunctioning venous valves, and thus, poor blood flow back towards the heart. Symptoms may include skin discoloration, dermatitis, venous ulcers, and hypoxia. In some cases, CVI can lead to secondary lymphedema. Secondary lymphedema is a result of a disruption of the lymphatic system (lymph nodes have been damaged or surgically removed). As lymphedema progresses, the affected area begins to swell as bodily fluids cannot be drained away. In the worst cases, the inflicted area requires reconstructive surgery to reduce swelling and remove damaged tissue. Compression therapy utilizing SMAs may be employed to help manage CVI and lymphedema symptoms with the additional benefit of heat therapy (which may be direct result of Joule heating). SMAs applications may include, for example, linear actuators in micro-positioning systems, bionic muscles in bio-inspired robotic animals, human use, and human use for external medical treatment.

The response of an SMA wire may be non-linear, hysterical, application-specific, and dependent on initial conditions such as pre-activation stress (pre-stress) and temperature. An SMA wire's deflection (strain) may be proportional to the percent of martensite (M) in the wire as it undergoes transformation. Thus, the SMA's wire's length may be proportional to the percent of martensite phase in the wire as it undergoes transformation. The percent of martensite or martensite-fraction may be calculated based on current wire temperature and known transformation temperatures. However, depending on the direction of transformation—martensite to austenite (M→A), or austenite to martensite (A→M)—the relationship between the martensite-fraction and strain, and between the martensite-fraction and load, may become offset by a hysteresis amount. Furthermore, the martensite-fraction may be dependent upon transformation temperatures, and transformation temperatures may be a function of the SMA wire's pre-stress.

As described herein, the stress-strain relationship of the actuating SMA may be linearly regulated by a restoring spring, where the linear slope of the stress-strain equation may be defined by the biasing spring and the stress axis intercept is defined by the initial pre-stress. In either the martensite or the austenite (A) phases, the resistance-strain relationship may be non-linear as the wire resistance appears to be more dependent on the temperature induced by Joule heating. The relationship between SMA wire strain and its resistance may display relatively small hysteresis compared to the relationship between SMA wire strain and its temperature. Ultimately, for linear position control using SMAs, the stress and strain may be largely inferred by monitoring the wire's resistance or its temperature if the initial pre-stress value is known. Resistance relationships before and after transformation may have a temperature dependence. The rate change of resistance may be independent of the SMA wire's pre-stress value and may be directly proportional to the induced wire temperature in the M-phase or A-phase; any change in the wire's pre-stress causes an absolute offset in the wire's resistance values. Moreover, the rate of change of resistance in the M-phase may be larger than the rate change in the A-phase.

An SMA may be used as bionic muscle, such as, for example to morph the wings of a bionic bat between folded and un-folded states (servo motors may be used to flap the unfolded wings). The SMA wires may be used as antagonistic linear actuators to flex or extend the robotic bat's elbow joint; these actuators are analogous to bicep or tricep muscles. When characterizing the SMA as a bionic muscle, a linear relationship was observed between the resistance of the SMA wire and the angle of the elbow joint. The defining slope of this relationship was dependent on the ambient temperature. An active, soft-orthotic application of SMAs may be used to treat and assist persons who suffer from neuromuscular disorders involving patients that have a noticeably obstructed gait.

In an example embodiment, an SMA may be cold-worked into a spring form and then annealed so that this spring form would be the “memorized” state. Multiple sets of springs may be placed behind a knee joint, or the like, such that their contraction would force knee flexion. Multiple sets of SMA springs may produce full knee flexion in a robotic leg; however, due to the passive cooling of the SMA springs, the frequency of actuation may be too low to replicate or assist in a cyclical walking gait. A potential solution to address this limitation is active cooling.

### Radial Compression Experiments

As described herein, experiments were conducted. The experiments focused, at least in part on relationships between radial forces, resistance, and temperature of SMA wires when they generate compression forces.

The SMA used in the experiments comprised FLEXINOL® wires. It is to be understood that any appropriate SMA material may be utilized for compression therapy as described herein, and the SMA material is not limited to FLEXINOL® wires. The SMA utilized in the experiments comprised a Nickle-Titanium SMA manufactured by Dynalloy, Inc. In the experiments, a 300 μm diameter SMA wire had a maximum actuation force of 41.6 N or an equivalent 4.24 F_{kg}. For the low-temperature wire type, transformation occurred when the wire was heated above 68 C. Normally, a FLEXINOL® wire actuation efficiency is specified as 5%, while the remaining 95% of the energy is dissipated as heat.

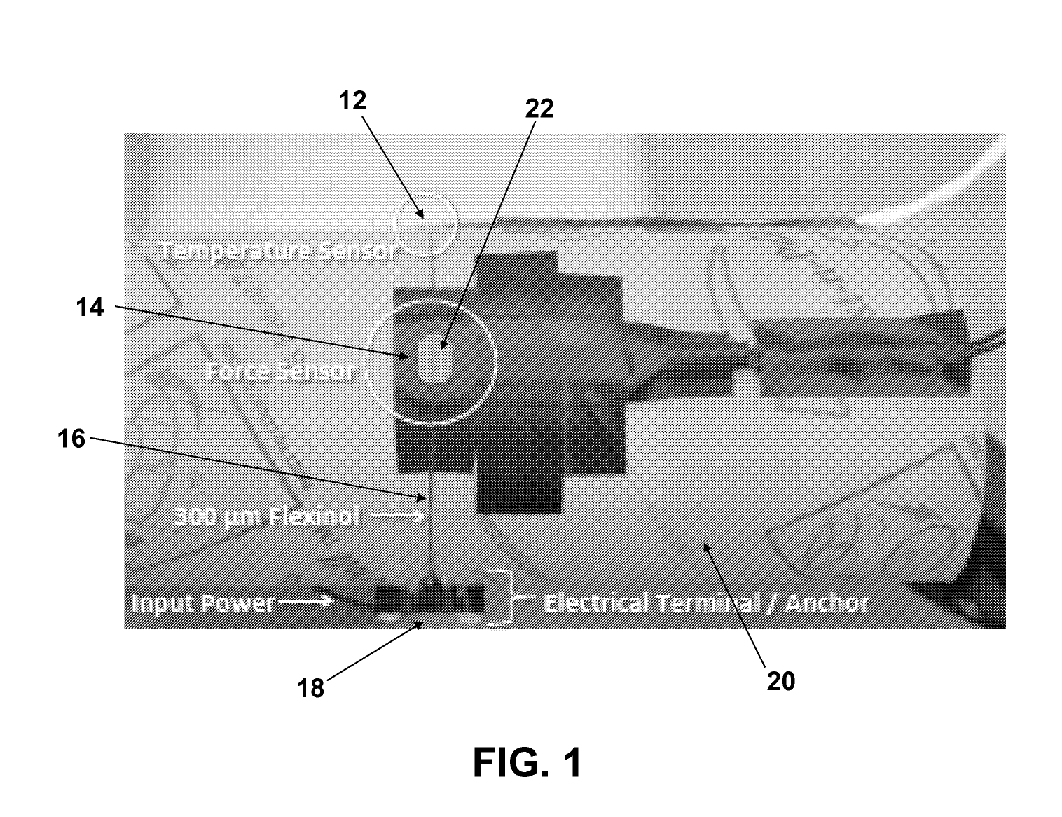

FIG. 1 depicts an example radial compression rig used for the experiments. As depicted in FIG. 1, the rig comprised a temperature sensor 12, a force sensor 14, an SMA wire 16, power supply 18, and a cylindrical cardboard tube 20. The cylindrical tube 20 was made of a thick cardboard that was stiff enough to avoid significant radial deformation as the SMA wire 16 actuated. Effectively, the tube 20 blocked the contraction of the SMA wire 16, thereby generating radial forces.

The SMA wire is depicted in FIG. 1, in the center of the tube 20 with an electrical screw terminal attached at either end of the SMA wire 16. These terminals were screwed onto the cylindrical tube 20 in order to keep the SMA wire 16 anchored. The length of SMA wire 16 was selected so that the SMA wire 16 lays flush to the cylinder 20 with minimal slack (≈23 cm). The SMA wire was not stretched before being anchored. Therefore, each test began with minimal pre-stress when the SMA wire 16 was in its complete martensite phase.

The force sensor 14 comprised a force sensitive resistor (not shown in FIG. 1). In the experiments, the force sensitive resistor comprised an Interlink FSR-402 Force-Sensitive Resistor (FSR). It is to be understood however, that any appropriate force sensor may be utilized for compression therapy as described herein, and the force sensor is not limited to an Interlink FSR-402 FSR. The force sensor 14 was placed under the SMA wire 16. A single layer of black tape was positioned between the SMA wire 16 and a plastic cutout 22. The cutout 22 served at least two purposes. First, the cutout 22 refocused the compressive force of the SMA wire 16 onto a larger portion of the force sensor 14. Second, because the force sensor 14 has a time dependent heat sensitivity, the cutout 22 also served as a thermal insulator. The plastic cutout 22 and the single layer of electrical tape anchored the force sensor 14 while further insulating the force sensor 14 from ambient temperature changes. Appropriate calibration methods were used to determine the tension of the SMA wire.

A temperature sensor 12 was positioned about 90 degrees behind the force sensor 14 on the outside of the tube 20 touching the SMA wire 16. In the experiments, the temperature sensor 12 comprised an AD590 temperature transducer. It is to be understood however, that any appropriate temperature sensor may be utilized for compression therapy as described herein, and the temperature sensor is not limited to an AD590 temperature transducer. The temperature sensor 12 was calibrated at room temperature.

### Instrumentation

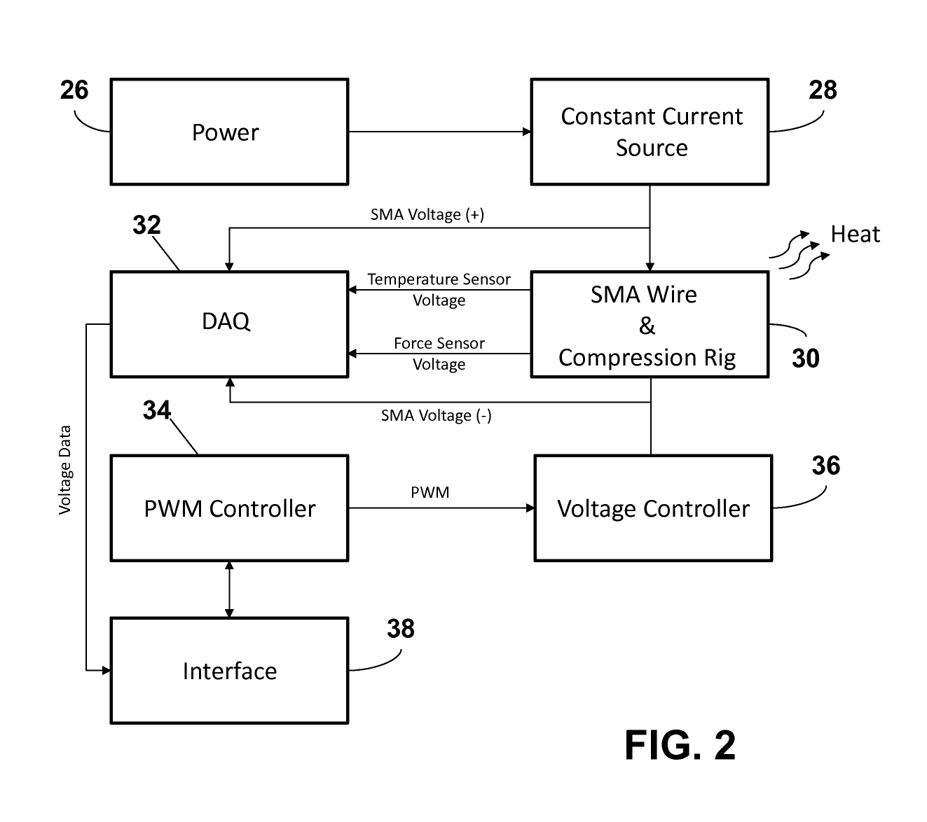

FIG. 2 depicts the overall instrumentation system used for the experiments. A power source 26 supplied constant power to the SMA Wire and Compression rig 30 during each test using a pulse-width-modulated (PWM) constant current source (CCS) 28. The CCS 28 supplied constant current independent of change in the SMA wire resistance. A National Instruments USB 6009 data acquisition device (DAQ) 32 was used to log multiple sensory inputs, while an Arduino Uno controller 34 was used to implement PWM changes. All components were interfaced interface 38 where calculations were performed and user commands were entered. For power control, the sampled differential voltage across the SMA wire was used to calculate its resistance and, ultimately, its power consumption. Based on the differential voltage value, a switching power MOSFET was used to regulate the time-averaged power, P_{SMA}, which can be described as:

P_{SMA}=I_{CCS}V_{SMA}−PWM/255

where I_{CCS} is the constant current value, V_{SMA} is the differential voltage across the SMA wire, and PWM/255 is the duty cycle of the PWM output. The majority of the resistance data was collected during the active powering of the SMA wire. When the SMA wire was allowed to passively cool, the differential voltage was sampled using a pulsed input lasting 800 μs with a period of 120 ms. This corresponds to a duty cycle of 0.67%, which has a negligible effect on the SMA wire's temperature and phase status.

To enhance system performance and accuracy, a series of differential voltage samples were first accumulated in the DAQ 32. After a 38 ms interval, the data was serially transmitted to the user interface 38 where the average value of each series of samples was used to determine the required duty cycle adjustment to maintain constant power. Each newly calculated duty cycle was then transmitted to the PWM controller 34 via serial communication. With an 8-bit PWM resolution, the constant power supply maintained a 5% regulation margin or better.

In order to make the test results more applicable to any length of 300 μm low-activation-temperature SMA wire, a Watt-per-Ohm parameter was defined. The total input power was thus scaled by the initially measured SMA wire resistance, which was specified as 13 Ω/m. This was a one-time initial calculation at the beginning of a single test when the wire was not actively powered.

### Experimental Testing Procedure

Six series of tests were conducted, each with different power-on times ranging from 0.5 seconds to 3 seconds in half-second increments. For each series, the input power was incremented by 200 mW/Ω over the range of 200 mW/Ω to 2.6 W/Ω. For each test, the SMA wire was powered 5 separate times and allowed to cool for 12 seconds after each power cycle. The SMA wire was not touched or moved during the entire testing procedure so as not to introduce inconsistencies due to wire placement or incidental stress. Before testing, the constant current levels of CCS 28 were checked while the control circuitry powered a dummy resistive load. During each test, SMA wire temperature, resistance, and radial force readings were recorded. After testing, all sampled data was processed to remove high frequency noise and transformed to respective units (° C., Ω, F_{Kg}).

The behavior of the SMA wire was observed by varying the input power value and input power-on time.

In the following, resistance and force results are reported for the input power levels of 0.4 W/Ω, 0.8 W/Ω, 1.2 W/Ω, 1.6 W/Ω, and 2.0 W/Ω where the power-on period is 2 seconds. The resistance and force trends for the remaining power-on periods (0.5 s through 3.0 s) were similar to those observed in the following figures, and are therefore not shown in the interest of brevity.

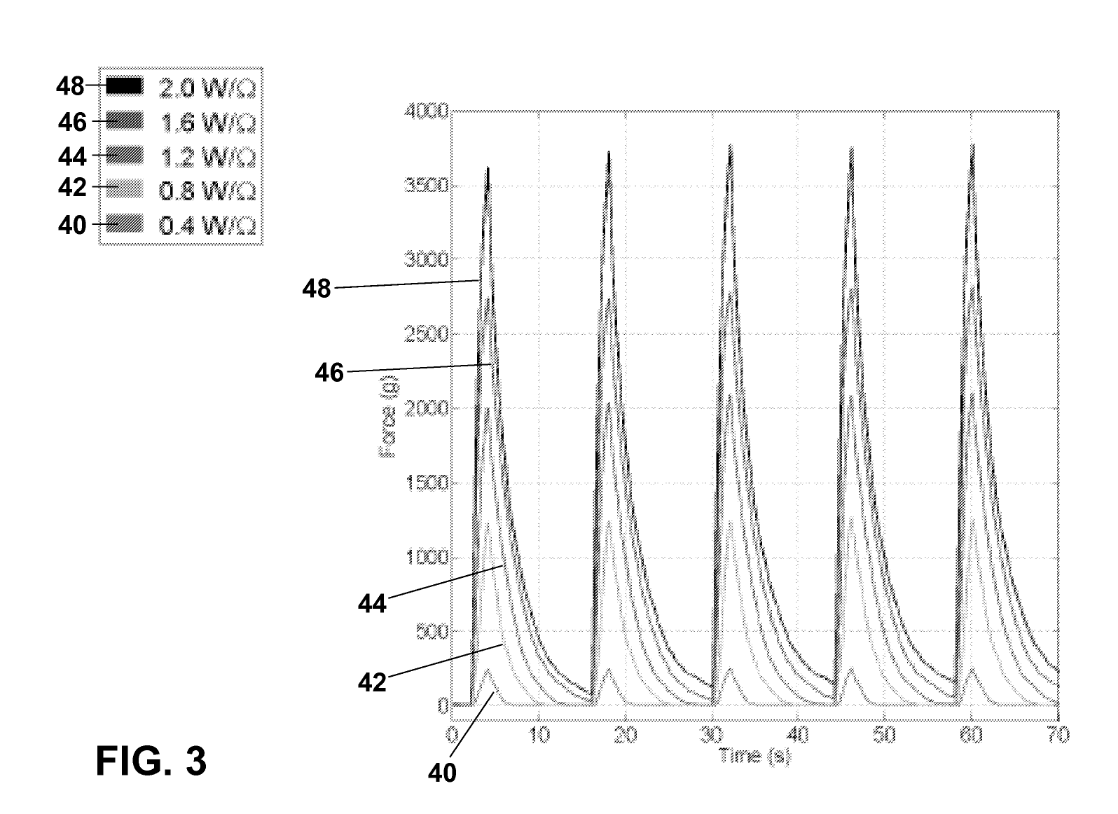

FIG. 3 depicts an example graphic illustration of radial force versus time for five power cycles. Curves representing force versus time are depicted for input power levels of 0.4 W/Ω (curve 40), 0.8 W/Ω (curve 42), 1.2 W/Ω (curve 44), 1.6 W/Ω (curve 46), and 2.0 W/Ω (curve 48) where the power-on period was 2 seconds and cool off time (power-off time) was 12 seconds. The resistance and force trends for the remaining power-on periods (0.5 s through 3.0 s) were similar to those observed in the following figures, and are therefore not shown in FIG. 3. For the 2-second power-on period when power was supplied, the force continually increased. Once power was removed, the force dropped off as an exponential decay. It was observed that with an increase in the input power levels, the time taken by the wire to generate a given force decreased. As seen in FIG. 3, the maximum forces are positively correlated with the maximum power, and monotonically increase with increasing power levels.

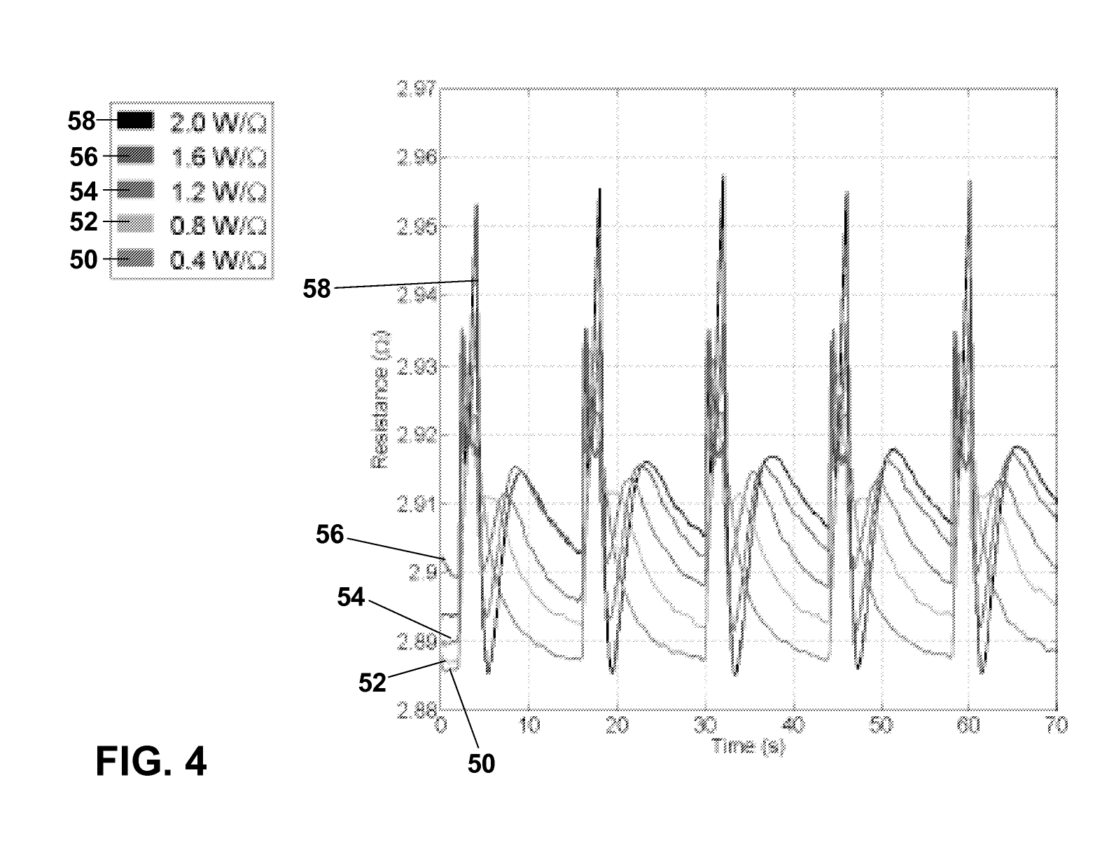

FIG. 4 depicts a graphic illustration of resistance versus time for five power cycles. Curves representing resistance versus time are depicted for input power levels of 0.4 W/Ω (curve 50), 0.8 W/Ω (curve 52), 1.2 W/Ω (curve 54), 1.6 W/Ω (curve 56), and 2.0 W/Ω (curve 58) where the power-on period was 2 seconds and cool off time (power-off time) was 12 seconds. As shown in FIG. 4, the resistance trends were repeatable.

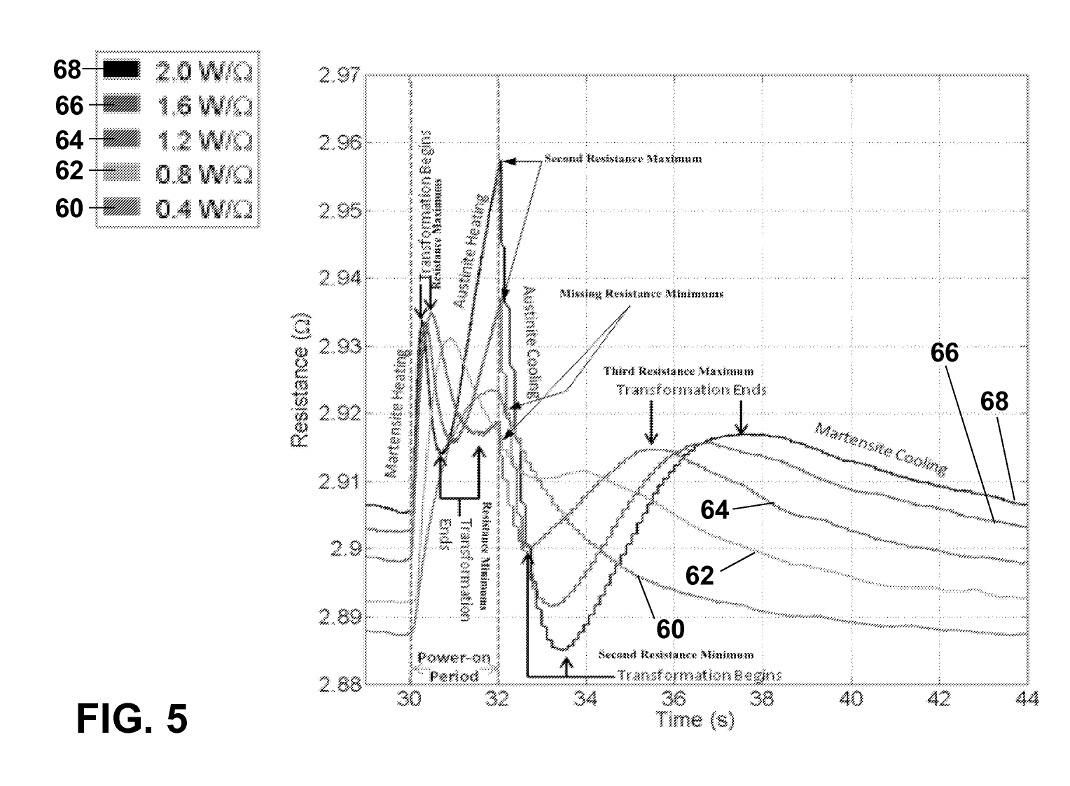

FIG. 5 depicts a graphic illustration of resistance versus time for a single power cycle. Curves representing resistance versus time are depicted for input power levels of 0.4 W/Ω (curve 60), 0.8 W/Ω (curve 62), 1.2 W/Ω (curve 64), 1.6 W/Ω (curve 66), and 2.0 W/Ω (curve 68) where the power-on period was 30 seconds and cool off time (power-off time) was 32 seconds. As shown in FIG. 5, the first maximum and minimum on the resistance plots correspond to the beginning and the end of the M→A phase transformation, respectively. For the 0.4 W/Ω input power level, the wire did not reach full phase transformation, as indicated by the missing minimum. For the curves that indicate transformation, the second maximum is the result of continuing to heat the wire. Once power is turned off, the wire undergoes the reverse A→M transformation as indicated by the second minimum and third maximum. The resistance begins to settle as the wire becomes sufficiently cool.

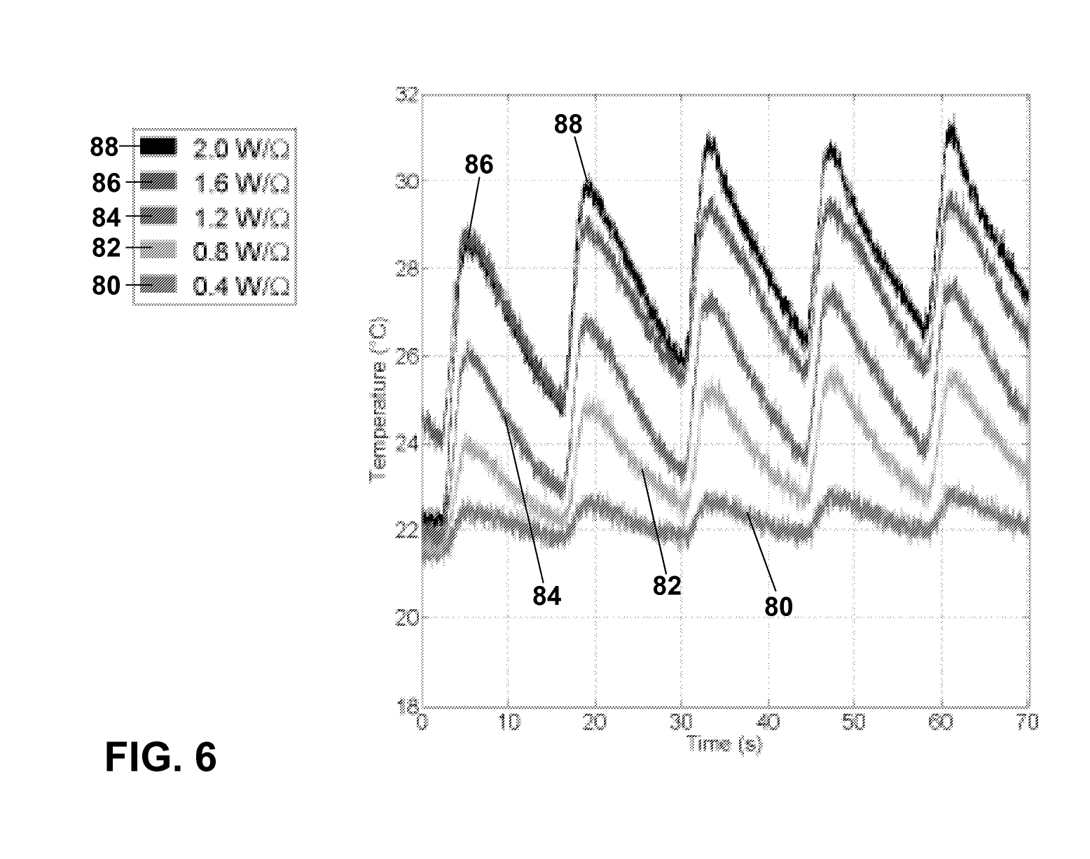

FIG. 6 depicts a graphic illustration of the effect of varying input power level on local temperature. Curves representing temperature versus time are depicted for input power levels of 0.4 W/Ω (curve 80), 0.8 W/Ω (curve 82), 1.2 W/Ω (curve 84), 1.6 W/Ω (curve 86), and 2.0 W/Ω (curve 88) where the power-on period was 2 seconds and cool off time (power-off time) was 12 seconds. The curves in FIG. 6 show the local temperature of SMA wire where the electrically isolated transducer touches the SMA wire. As depicted in FIG. 6, a positive correlation between input power and rate change of temperature, as well as maximum temperature are observed.

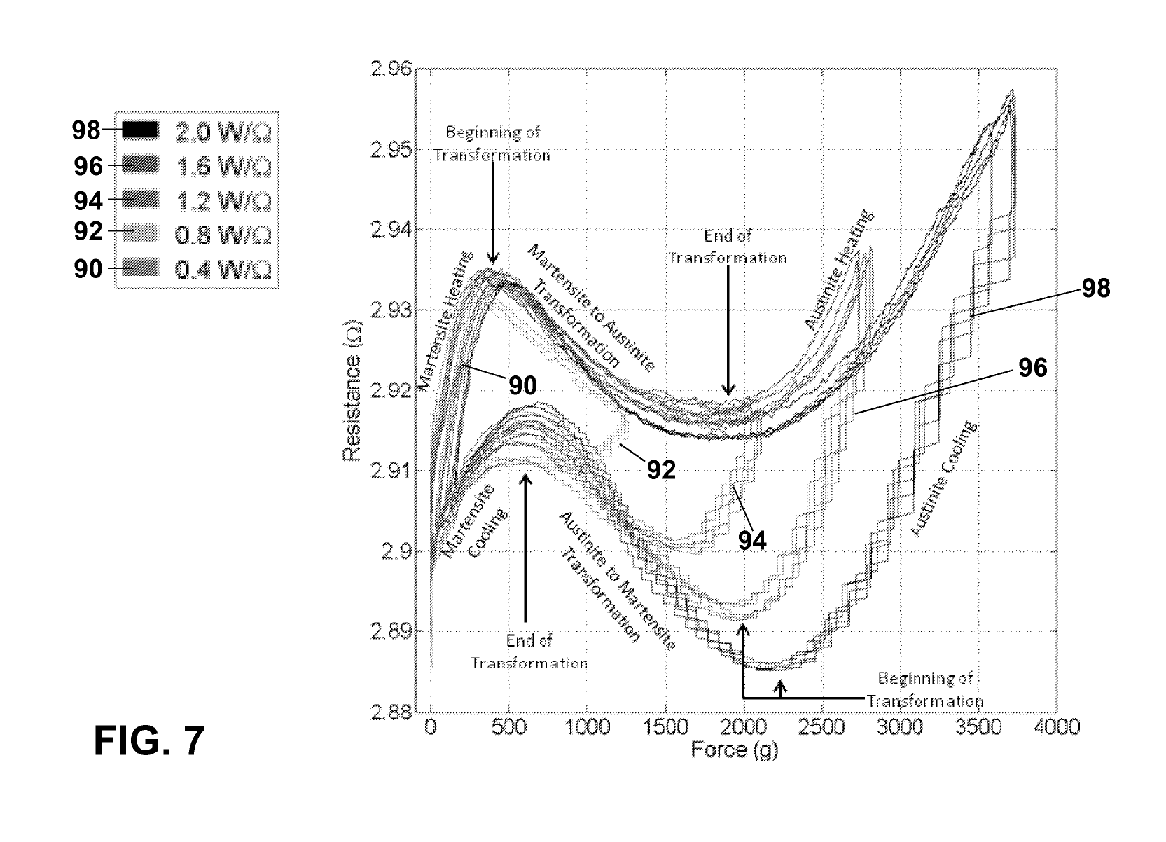

FIG. 7 depicts a graphic illustration of resistance versus force. Curves representing resistance versus force are depicted for input power levels of 0.4 W/Ω (curve 90), 0.8 W/Ω (curve 92), 1.2 W/Ω (curve 94), 1.6 W/Ω (curve 96), and 2.0 W/Ω (curve 98) where the power-on period was 2 seconds and cool off time (power-off time) was 12 seconds. Resistance is plotted on the vertical axis to show the similarity to the time-dependent resistance plot shown in FIG. 5. FIG. 7 shows the hysteresis relationship between force and resistance. The upper branch of FIG. 7 represents the power-on portion, and the lower branch represents the power-off (cool down) portion, and together form the hysteresis response of the SMA wire. The maximums and minimums indicate phase transitions and show that approximately half of the force is generated in the A-phase.

If the resistance-force plot of FIG. 7 is viewed similarly to FIG. 5, the M-phase, M/A-phase, and A-phase become apparent by noting the maximum and minimums as transformation indicators. The curves of FIG. 7 show that almost half of the induced wire stress or radial force generation, approximately 280 MPa (≈2000 g over a 300 μm cross section wire), is produced in the Austinite-phase. This may due, at least in part, because while the Joule heating produces the phase transformation, the cylindrical tube acts to block the SMA wire from completely transforming to its unstressed austenite length (5% strain). As the SMA wire continues to get hotter, more energy is available to assist in its recovery, generating more radial force.

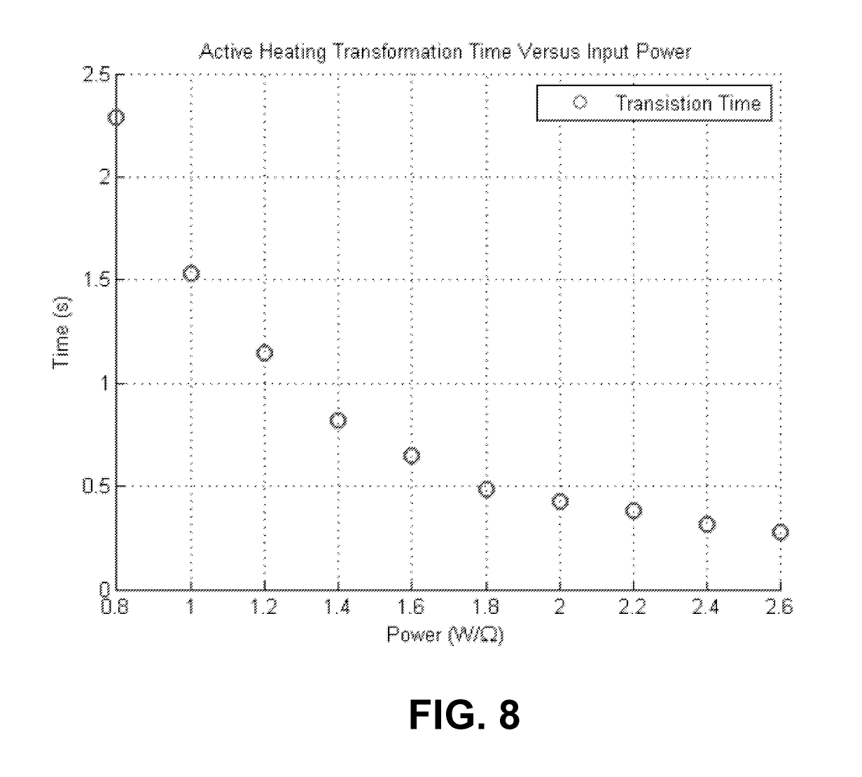

FIG. 8 depicts a graphic illustration of phase transformation time versus input power. FIG. 8 depicts the change in the local temperature over time. Transformation (transition) times decreased as input power increased. As described herein, phase transformation time is a metric used to specify the time response of the actuating wires. The circles represent transition time on the graph. FIG. 8 shows the M→A transformation times for the 300 μm SMA wire under various input power levels. The plot of FIG. 8 shows an approximate two-fold reduction in transformation time for a 50% increase in power. Overall, for increasing input power, the decrease in transformation time becomes marginalized.

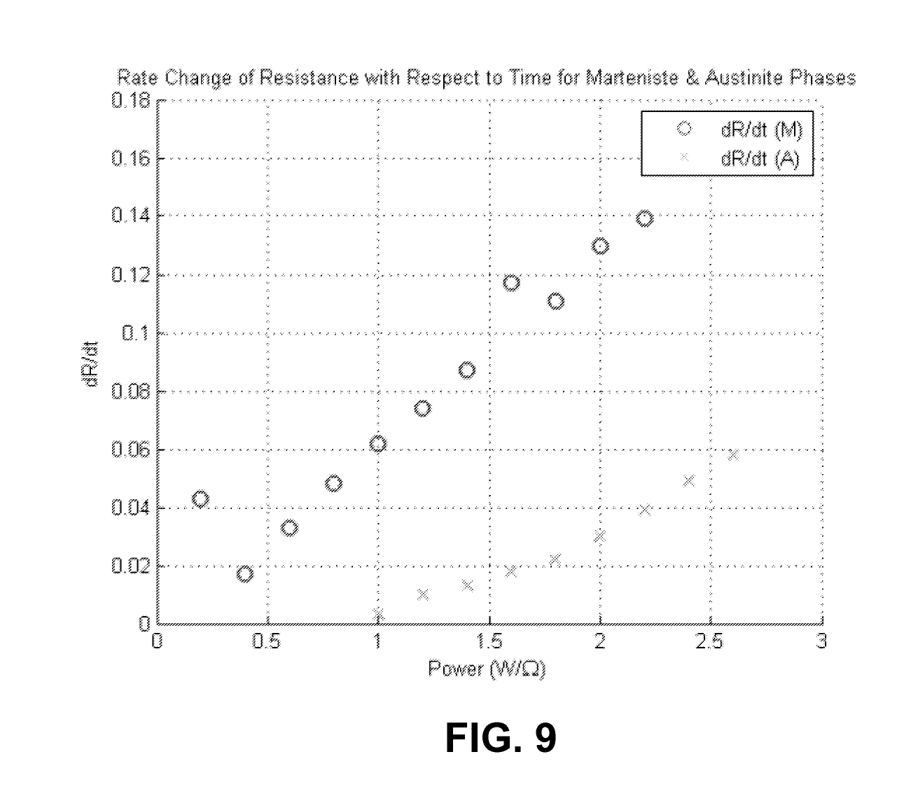

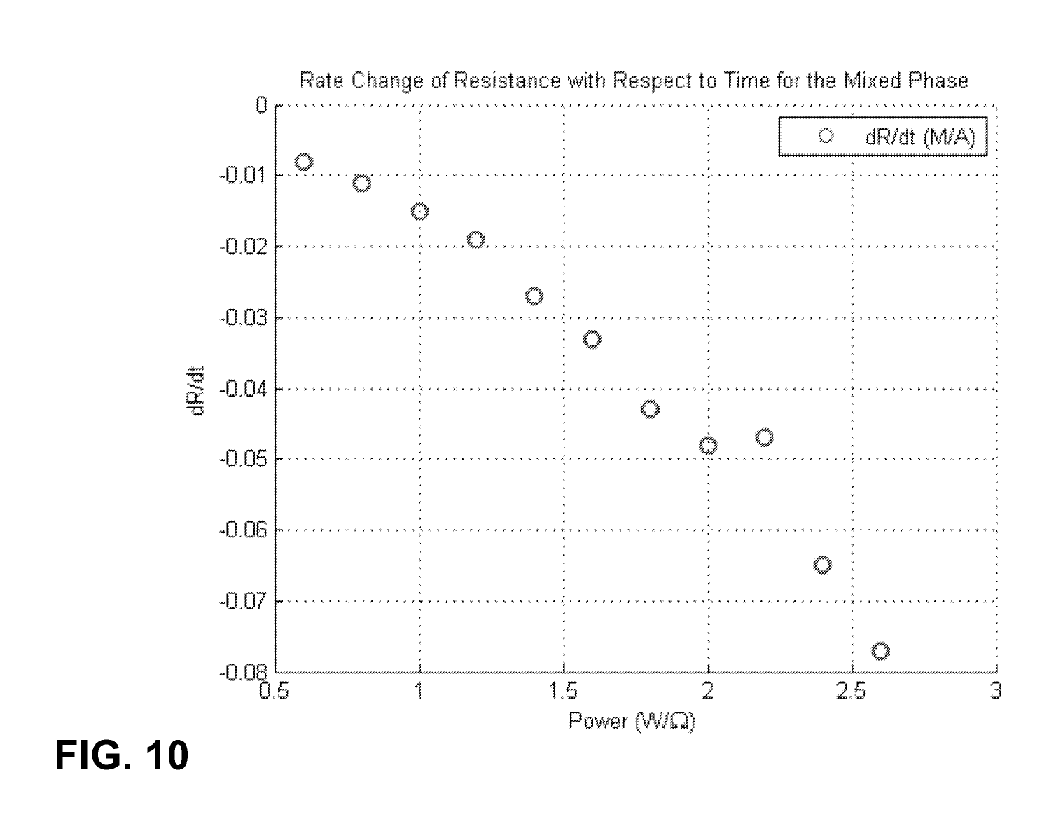

FIG. 9 depicts a graphic illustration of the rate of change of wire resistance during the mixed phase (M/A) period for different input power levels. In addition to looking at the transformation time, the rate of change of wire resistance may provide information about the dynamic response of the actuating wires. The time-derivative of resistance under different power settings is shown in FIG. 9 for both the M-phase and A-phase, and in FIG. 10 for the M/A-phase period. These plots show data for the active heating portion. The M-phase has a much higher rate change than the austenite. Furthermore, except for two outlying points on the martensite curve, the relationship between rate of change of wire resistance and the input power is almost linear (FIG. 9). Similarly, in the mixed phase state, the input power and the absolute rate change of resistance are negatively correlated (FIG. 10). As mentioned earlier, the SMA wire did not finish transformation for the 0.2 W/Ω to 0.8 W/Ω range, and hence, there are no data points for these cases.

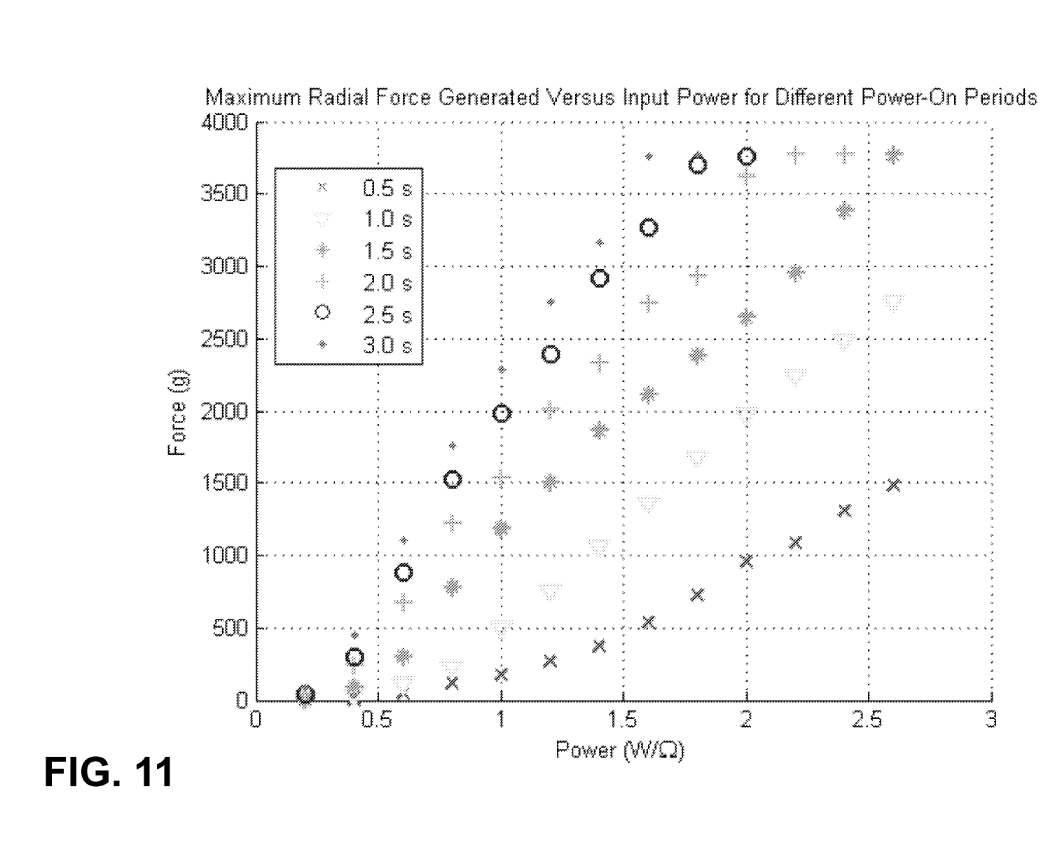

FIG. 11 depicts a graphic illustration of maximum (peak) attainable force versus input power for different power-on times. The absolute maximum observed force, as limited by the operating range of the force sensor amplifier circuit, is visible in FIG. 11 at approximately 3750 grams of force. It is observed from FIG. 11 that for a given power level, the maximum attainable radial force increases with an increase in the input power-on times. FIG. 11 also shows that for the same total input energy, the maximum observed forces for different input power levels are not equivalent. This observation also relates to the observed local temperature as seen in FIG. 6. The maximum temperatures achieved are directly proportional to the input power and duration of supplied power. Essentially, more energy must be spent for a lower input power than for a higher input power when trying to obtain the same maximum force.

The foregoing describes radial compression response when an object under compression experiences minimal radial deformation. Neither ambient temperature nor insulate of SMA wire was controlled. The following results were observed.
1) The A-phase generates almost half of the maximum observed force of 3750 grams.
2) A positive correlation relationship exists between input power for a given power-on period and maximum force.
3) The time-dependent rate change of resistance is higher in the M-phase than in the A-phase.
4) There is a two-fold reduction of transformation time for a 50% input power increase.
5) There is a positive correlation between input power and rate change of resistance in the M-phase and the A-phase.

The foregoing description has examined the characteristic response of a 300 μm shape-memory-alloy wire, as it was electrically activated when wound around a rigid cylindrical tube. This activation gave rise to a distributed radial force, which has been measured in the relative form of the SMA wire's tension. It was noted that the force exerted by the SMA wire for a given activation period was positively correlated to the input power. Also, an examination of the force and resistance trends showed that even after the wire transformed to the austenite phase, the exerted force still continued to increase as the wire was powered. Overall, results were repeatable for multiple cycles. Furthermore, several characteristic trends have been discussed and compared to other studies where SMA wire was used for linear actuation. Applications may be directed to SMAs comprising softer material that allow for radial deformation.

Additional radial compression experiments were conducted focusing the relationship between input power, radial forces, resistance, and local temperature of SMA wires when subjected to radial actuation.

As noted, the response of a SMA wire may be non-linear, hysterical, application-specific, and dependent on initial conditions such as pre-activation stress (pre-stress) and temperature. The wire's deflection (strain) may be proportional to the percent of martensite (M) of the wire as it undergoes transformation. The percent of martensite or martensite-fraction may be calculated based on current wire temperature and known transformation temperatures. While the martensite-fraction may be dependent on the transformation temperatures, the transformation temperatures may, in turn, be a function of the wire's pre-stress.

In the following described experiments, the SMA wire response was analyzed under linear actuation and it was observed that the relationship between SMA wire resistance and deflection in the Mixed-martensite (M/A) phase was nearly linear. For this application, the stress-strain relationship of the actuating SMA was linearly regulated by a restoring spring, where the slope of the (linear) stress-strain equation is defined by the biasing spring and the stress axis intercept is defined by the initial pre-stress. In either the martensitic or the austenite (M or A) phases, the resistance-strain relationship was non-linear as the wire resistance appeared to be more dependent on the temperature induced by Joule heating. The relationship between SMA wire strain and its resistance displayed relatively small hysteresis compared to the relationship between SMA wire strain and its temperature. Ultimately, for linear position control using SMAs, the stress and strain may be largely inferred by monitoring the wire's resistance or its temperature if the initial pre-stress value is known.

As observed in the following described experiments, resistance relations before and after transformation may have a large temperature dependence. The linear rate change of resistance that accompanies temperature change for linear actuation applications was measured. It was found that the rate change of resistance is independent of the wire's pre-stress value and is directly proportional to the induced wire temperature in the M-phase or A-phase; and any change in the wire's pre-stress causes an absolute offset in the wire's resistance values. Moreover, the rate of change of resistance in the M-phase was significantly larger than the rate change in the A-phase.

Also described herein is the use of SMAs to morph the wings of a bionic bat between folded and un-folded states (servo motors are used to flap the unfolded wings). The SMA wires were used as antagonistic linear actuators to flex or extend the robotic bat's elbow joint. When characterizing the SMA as a bionic muscle, a well-defined linear relationship between the resistance of the SMA wire and the angle of the elbow joint was observed. And, in this case, the defining slope of this relationship was dependent on the ambient temperature.

Also described herein is an active, soft-orthotic application for treating and assisting persons who have obstructed walking gaits resulting from neuromuscular disorders, or the like. The SMA wire was first cold-worked into a spring form and then annealed so that this spring form would be the “memorized” state. Multiple sets of springs were placed behind the knee joint such that their contraction would force knee flexion. It was concluded that four sets of SMA springs could produce full knee flexion in a robotic leg. However, due to the passive cooling of the SMA springs, the frequency of actuation was too low to replicate or assist in a cyclical walking gait. A potential solution to address this limitation is active cooling. Different active cooling methods that increase the maximum actuation frequency of SMA wires are described.

The structure of an SMA may be defined by two main phases: martensite and austenite; both dictated by internal wire temperature. In martensite phase, which exists at lower temperatures, the SMA is relatively soft and easily deformed. Austenite, which occurs at higher temperatures, is indicated by a stronger and cubic structure in the SMA. The end of the martensite phase may be defined as the point when resistance change due to shape change exceeds that due to heating. Both a “Mixed” phase and “Pseudo-austenite” phase are used in explaining the data and wire behavior in this paper. The mixed (M/Pseudo-A) phase may be used to describe when the wire structure is in a state between martensite and Pseudo-austenite. Pseudo-austenite (Pseudo-A) may begin when the SMA wire's resistance due to shape change decreases enough to be exceeded by the change in wire resistance from heating.

The SMA used in the experiments comprised FLEXINOL® wires. It is to be understood that any appropriate SMA material may be utilized for compression therapy as described herein, and the SMA material is not limited to FLEXINOL® wires. The SMA utilized in the experiments comprised a Nickle-Titanium SMA manufactured by Dynalloy, Inc. In the experiments, a 300 μm diameter SMA wire had a maximum actuation force of 41.6 N or an equivalent 4.24 F_{kg}. The maximum strain was specified at 8%, while normal operation was suggested at 3% to 5% strain. For the low-temperature wire type, transformation occurred when the wire was heated above 68° C. Normally, a FLEXINOL® wire actuation efficiency is specified as 5%, while the remaining 95% of the energy is dissipated as heat.

### Physical Setup

The radial compression rig used for the following described experimentation was the same as previously described and shown in FIG. 1. Because the tube 20 blocks the contraction of the SMA wire 16, radial forces are generated.

The system used to conduct the following described experiments was the same as described above and shown in FIG. 2. And the system was operated in the same manner as described above. The majority of the resistance data was collected during the active powering of the SMA wire. When the wire was allowed to passively cool, the differential voltage was sampled using a pulsed input lasting 800 μs with a period of 120 ms. This corresponds to a duty cycle of 0.67%, which has a negligible effect on the wire's temperature and phase status.

### Experimental Testing Procedure

The testing procedure was the same as previously described. That is, six series of tests were conducted, each with different power-on times ranging from 0.5 seconds to 3 seconds in half-second increments. For each series, the input power was incremented by 200 mW/Ω over the range of 200 mW/Ω to 2.6 W/Ω. For each test, the SMA wire was powered 5 separate times and allowed to cool for 12 seconds after each power cycle. The SMA wire was not touched or moved during the entire testing procedure so as not to introduce inconsistencies due to wire placement or incidental stress. Before testing, the constant current levels of CCS 28 were checked while the control circuitry powered a dummy resistive load. During each test, SMA wire temperature, resistance, and radial force readings were recorded. After testing, all sampled data was processed to remove high frequency noise and transformed to respective units (° C., Ω, F_{Kg}).

The behavior of the SMA wire was observed by varying the input power value and input power-on time.

For the 2-second period when power was supplied, the force continually increased. Once power was removed, the force exponential decayed. It was observed that with an increase in the input power levels, the time taken by the wire to generate a given force decreased.

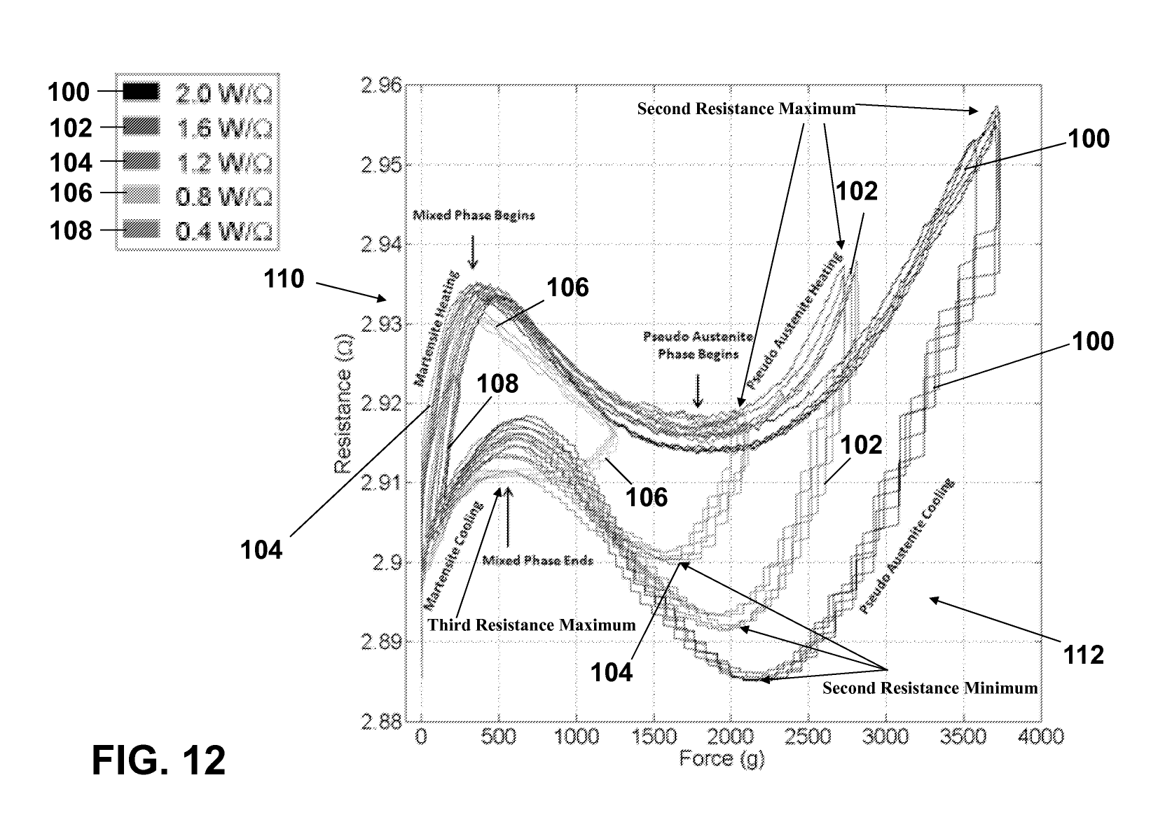

FIG. 12 is an example graph depicting resistance vs. force for five power cycles. Curves are depicted for input power levels of 0.4 W/Ω (curve 108), 0.8 W/Ω (curve 106), 1.2 W/Ω (curve 104), 1.6 W/Ω (curve 102), and 2.0 W/Ω (curve 100) where the power-on period was 2 seconds and cool off time (power-off time) was 12 seconds. The first maximum and minimum on the resistance plots correspond to the beginning and the end of the M/Pseudo-A-phase, Mixed phase, transformation, respectively. For the 0.4 W/Ω input power level, the wire did not reach complete Pseudo-A-phase transformation, as indicated by the missing minimum. For the curves that indicate transformation, the second maximum is the result of continuing to heat the wire. Once power was turned off, the wire underwent the reverse Pseudo-A/M transformation as indicated by the second minimum and third maximum. The resistance began to settle as the wire became sufficiently cool.

Referring again to FIG. 12, the upper branch 110 was the power-on portion, while the lower branch 112 was the cool-down portion, which, together formed the hysteresis response of the SMA wire. The maximums and minimums indicate phase transitions and show that approximately half of the force is generated in the Pseudo-A-phase. FIG. 12 illustrates the hysteresis relationship between force and resistance. Resistance is plotted on the vertical. The M phase, M/Pseudo-A-phase, and Pseudo-A phase become apparent by noting the maximum and minimums as transformation indicators. FIG. 12 indicates that almost half of the induced wire stress or radial force generation, approximately 280 MPa (≈2000 g over a 300 μm diameter wire), was produced in the Pseudo-A-phase.

The change in duration of the M/Pseudo-A-phase as input power was applied was as depicted in FIG. 8, and as explained herein with respect to FIG. 8.

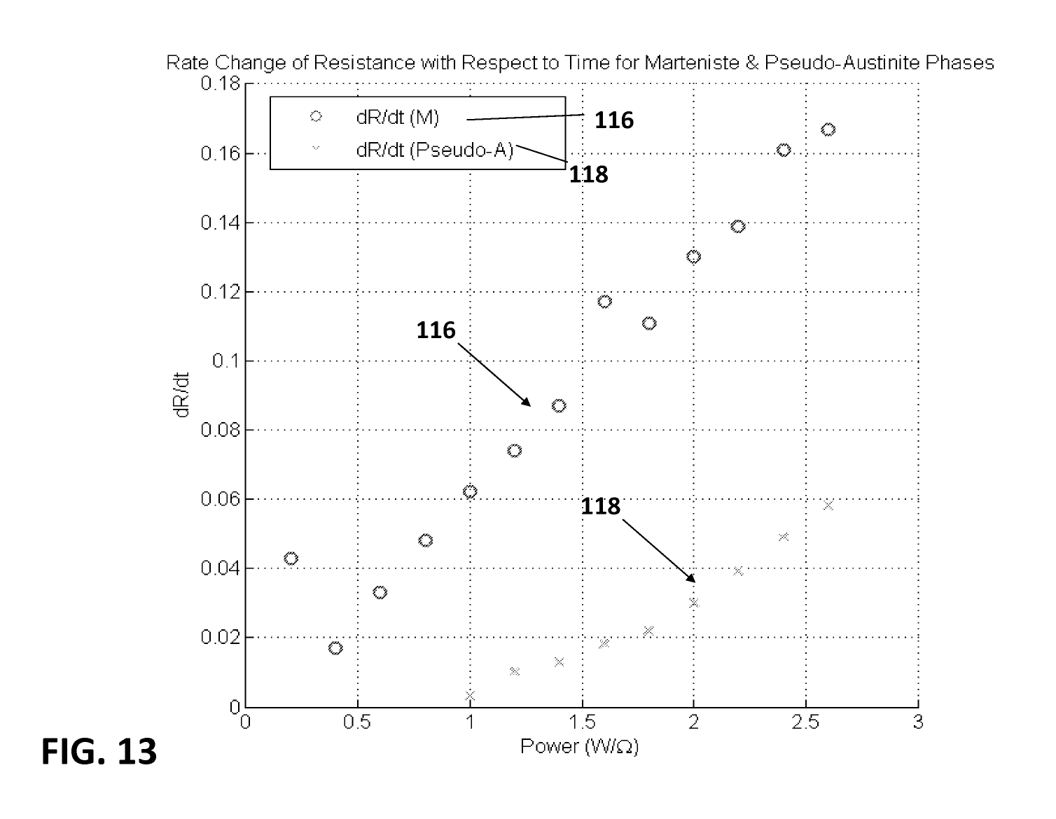

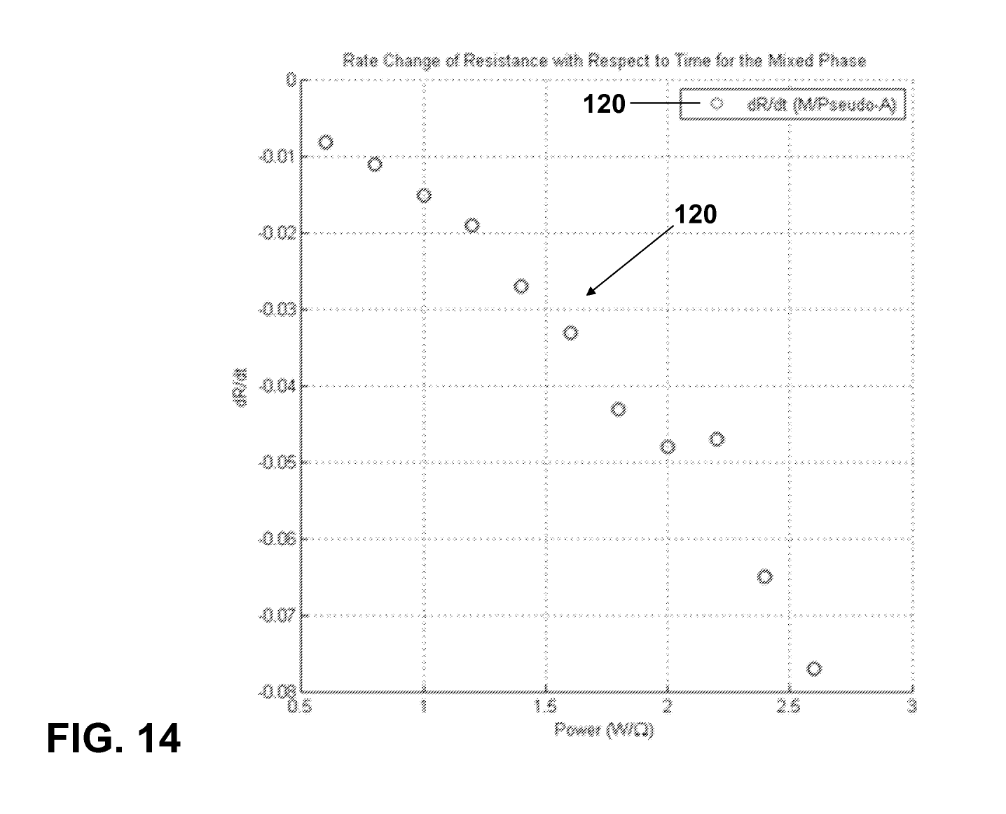

FIG. 13 is an example graph depicting the rate of change of resistance with respect to time under different power settings for both the M-phase (curve 116) and Pseudo-A-phase (curve 118). FIG. 14 is an example graph depicting the rate of change of resistance with respect to time under different power settings for the mixed phase, M/Pseudo-A-phase (curve 120). The rate of change of wire resistance may provide information about the dynamic response of the actuating wires. The graphs 116, 118, of FIG. 13 illustrate the derivative of SMA wire resistance with respect to time vs. power in watts per ohm. The graph 120 of FIG. 14 illustrates the derivative of SMA wire resistance with respect to time vs. power in watts per ohm. The plots 116, 118, 120, of FIG. 13 and FIG. 14 illustrate data for the active heating portion. The M-phase (curve 116) had a much higher rate change than the Pseudo-A-phase (curve 118). Except for two outlying points on the martensite curve 116, the relationship between rate of change of wire resistance with respect to time and the input power is almost linear. Similarly, in the mixed phase state (curve 120), the input power and the rate of change of resistance are negatively correlated. Pseudo-A-phase data points for power inputs lower than 1 W/Ω are not shown in FIG. 13.

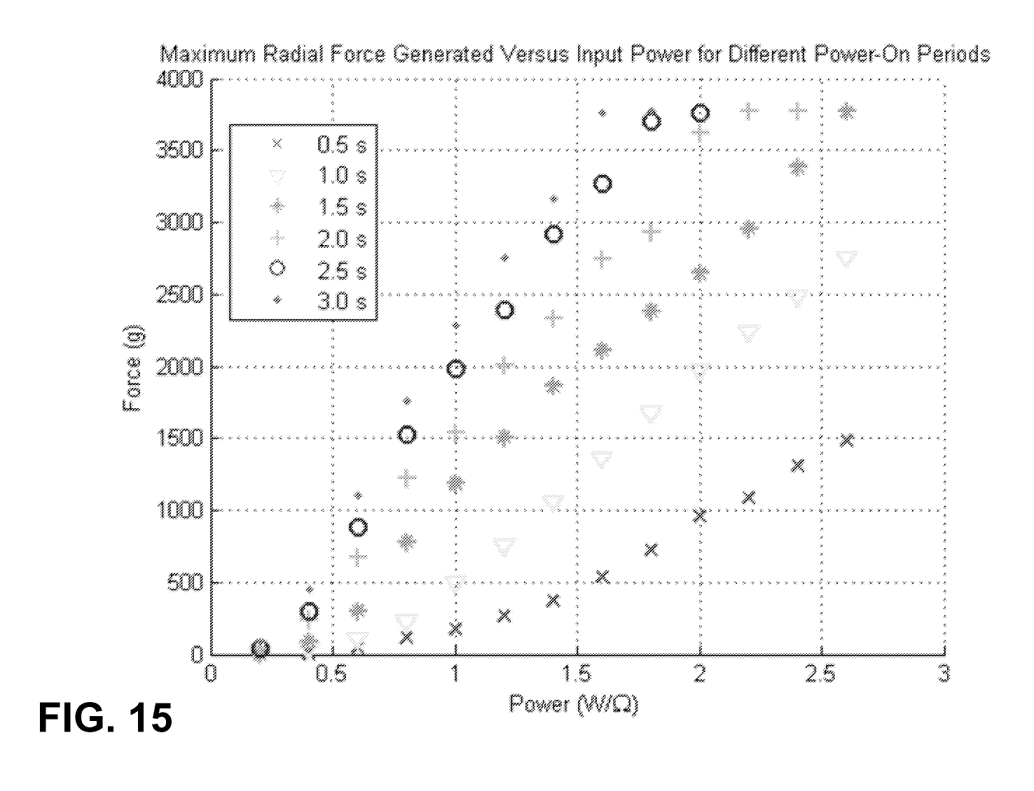

FIG. 15 is an example graph depicting the maximum force vs. power for different power-on times. The absolute maximum observed force, limited by the operating range of the force sensor, is visible in FIG. 6 as approximately 3750 grams. FIG. 15 also indicates that for a given power level, the maximum radial force increased with an increase in the input power-on time. FIG. 15 further indicates that for the same total input energy (power over a given time duration), the maximum observed forces for different input power levels were not equivalent.

The foregoing describes radial compression response when an object under compression experiences minimal radial deformation. Neither ambient temperature nor insulate of SMA wire was controlled. The following results were observed.

The foregoing described experiments examined the characteristic response of a 300 μm shape-memory-alloy wire as it was electrically activated when wound around a rigid cylindrical tube. This activation gave rise to a distributed radial force, which has been measured in the form of the wire's tension. It was noted that the force exerted by the SMA wire for a given activation period was positively correlated to the input power. Also, an examination of the force and resistance trends showed that even after the SMA wire transformed to the Pseudo-Austenite phase, the exerted force still continued to increase as the wire was powered. Results were repeatable for multiple cycles. Furthermore, several characteristic were observed.

Unlike the linear actuation case, a significant amount of force was generated in the Pseudo-A-phase when the wire is subjected to radial actuation. The Pseudo-A-phase generated almost half of the maximum observed force of 3750 grams. There was a positive correlation between input power for a given power-on period and maximum force. The rate change of resistance was significantly higher in the M-phase than in the Pseudo-A-phase. This is similar to the linear actuation case. There was a two-fold reduction of transformation time for a 50% input power increase. There was a positive correlation between input power and rate change of resistance in the M-phase and the Pseudo-A-phase.

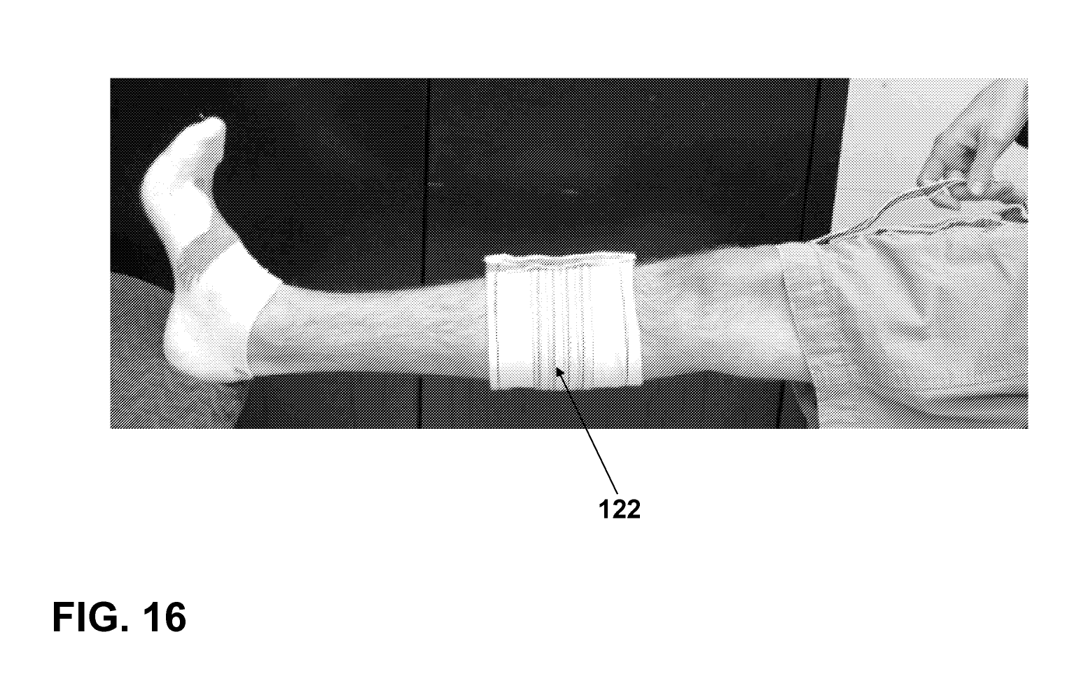

A SMA-based massaging sleeve prototype was constructed. A depiction of the prototype sleeve 122 is shown in FIG. 16. The prototype sleeve 122 comprised four separately controlled SMA wires. This sleeve 122 was controlled with a variety of electronic hardware components that were interfaced with a C# generated Graphical User Interface (GUI). The sleeve 122 was utilized to provide a demonstration massage on a dummy-arm with user-defined massage parameters that included compression force, compression duration, and general massage speed. Proof of functionality was provided in real-time feedback and post run data plots.

A SMA-based massaging sleeve may take advantage of the properties of shape-memory-alloy materials to provide concurrent heat and compression for recreational massage and/or medical compression therapy applications. Furthermore, a segmented massaging sleeve, as described herein, may allow for more focused compression while offering customized massage routines.

A factor considered in the design and construction of the SMA actuated system was the characterization of the SMA wire utilized. Therefore, the design of the sleeve and the characterization of the SMA were coupled. For an electrically controlled SMA system using Joule heating, the SMA wire itself may be used as a sensor. From real-time resistance information, information on the initial pre-stress of the wire and phase-state may be extracted. Depending on wire dimensions, and application, the characteristic response may change; thus, generally, for each wire type and application, a separate characterization may be needed. Multiple characterizations were performed during the design project.

In an example embodiment, multiple SMA wires may be individually actuated and perform independently from each other. The wires may be wrapped around the “limb” or the object to be compressed and may be fastened with an anchoring element. The anchoring may ensure the wire's contraction will be blocked by the object and ultimately result in a radial compression of the object as the wire tries to contract in length. The compression force from the wire may be distributed as an average pressure over a larger area. This pressure may be calculated as the measured axial force induced in the activated wire over the surface area, where this force is evenly distributed. In an example embodiment, one compressed segment may have a negligible effect on the adjacent segments.

The following are analyzed and described herein: (1) a functioning prototype sleeve, (2) controlled compression force, (3) variable compression duration, (4) overall massage speed, (5) a user interface for control, and (6) real time plots of the messaging sleeve performance.

In place of a person, a cylindrical testing rig was utilized. This rig offered radial symmetry as well as simplicity to the analysis since any non-ideal compression behavior such as active muscle flexing would not play a role in wire behavior. In taking the place of a human limb, the testing rig was constructed to be arm-like. Therefore, the testing rig material was comparable to human fat-muscle tissue. This was accomplished with the use of a medium-density ¼-inch foam sheet wrapped around the cylindrical rig.

Compression forces were measured without disturbing the radial symmetry inherent in the cylindrical rig setup. Thus, thin force sensitive resistors (FSRs) were used to measure the compression force. Before using the compressible foam on the testing rig, however, the FSRs were calibrated because they experienced some warping when they were pushed into the foam layer. In an example embodiment, single or multiple small air-bladders that can directly measure changes in pressure and are less affected by any irregular deformation that occurs beneath the SMA wire compression regions may be used.

Described below are the electronic hardware utilized, the prototype sleeve construction, the implemented user interface, the implemented control algorithms, and performance analysis.

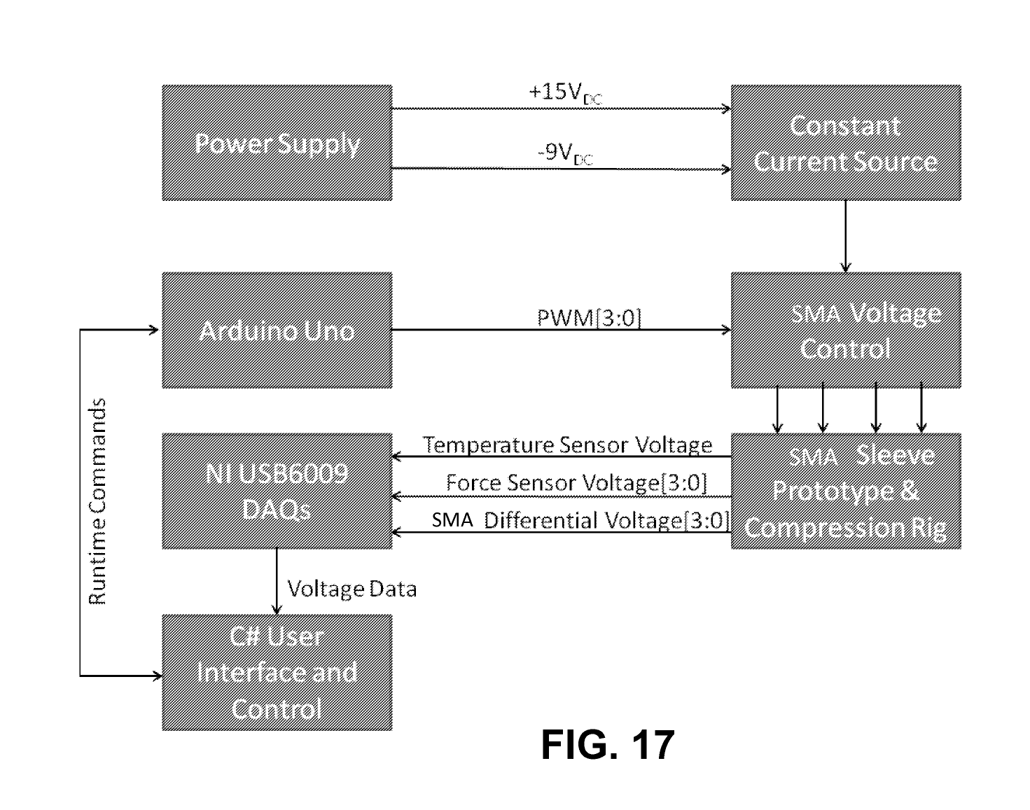

FIG. 17 depicts the overall system block diagram. Electronics hardware included two linear voltage regulators used in a constant current supply (CCS) configuration, four force sensor circuits utilizing inverting op-amp configurations and FSRs, one temperature sensing circuit, one Arduino Uno board, and two NI USB6009 data acquisition devices (DAQs). The CCS setup gave consistency and accuracy measurements of the wire's behavior, particularly the wire's resistance, and in the ability to manipulate and maintain the input power. The SMA wire exhibited a dynamic load whose resistance changed during activation with a range that was proportional to the length of wire. Thus, manipulating input power (instead of voltage or current alone) simplified behavior analysis. Also, regulating power helped to linearize the compression force response.

Regulation of power was achieved by pulse width modulating (PWM-ing) the input current via solid state relays (SSRs). The PWM duty cycle value was determined based on the measured differential voltage across each SMA wire and the desired input power. Indicator LEDs were placed in series with the SSR input terminals to indicate when the SSRs are on.

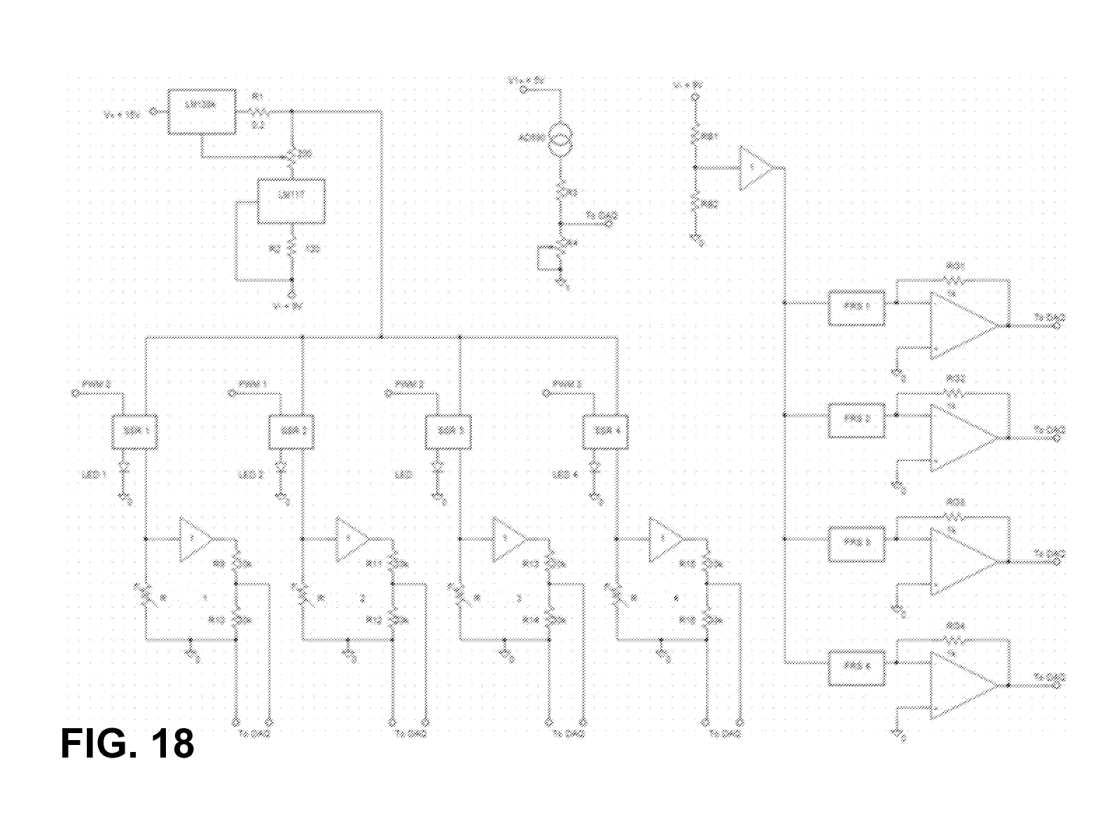

FIG. 18 is an example schematic diagram of the power regulation system and sensory equipment, wherein SSR represents a Solid State Relay, FSR represents a Force Sensitive Resistor, AD590 represents a temperature sensor, LM138k represents a voltage regulator for constant current, and LM117 represents a voltage regulator for reference voltage to LM138k.

The inverting op-amp configuration for the force sensors allowed for a more linear response from the measured FSR voltage (the conductance to force relationship in the FSR is approximately linear). A unity gain amplifier also was used in conjunction with a voltage dividing potentiometer that allowed for FSR gain adjustment by directly manipulating the negative input voltage. There was one temperature sensor circuit that used an AD590 semiconductor temperature sensor. While this sensor was calibrated for room temperature, noise was observed in temperature data as the sensor outputs 1 μA/° K, which translates to a few hundred millivolts of voltage. In an example embodiment, a differential amplifier stage may be utilized to boost the signal as well as reject common mode noise.

In regards to input/output (I/O), in this example embodiment, each SMA wire, FSR, and the temperature sensor was allocated a separate single ended channel. Two DAQs were used to increase the sampling frequency available for each channel.

The example embodiment of the sleeve prototype comprised a white cotton flannel fabric base, four 12″×⅜″× 3/32″ fire-resistant rubber strips, four 14″ lengths of 300 μm SMA wires, four ⅜″ aluminum studs, one piece of solderable prototyping board, and five 3-foot lengths of 22AWG solid-core wires.

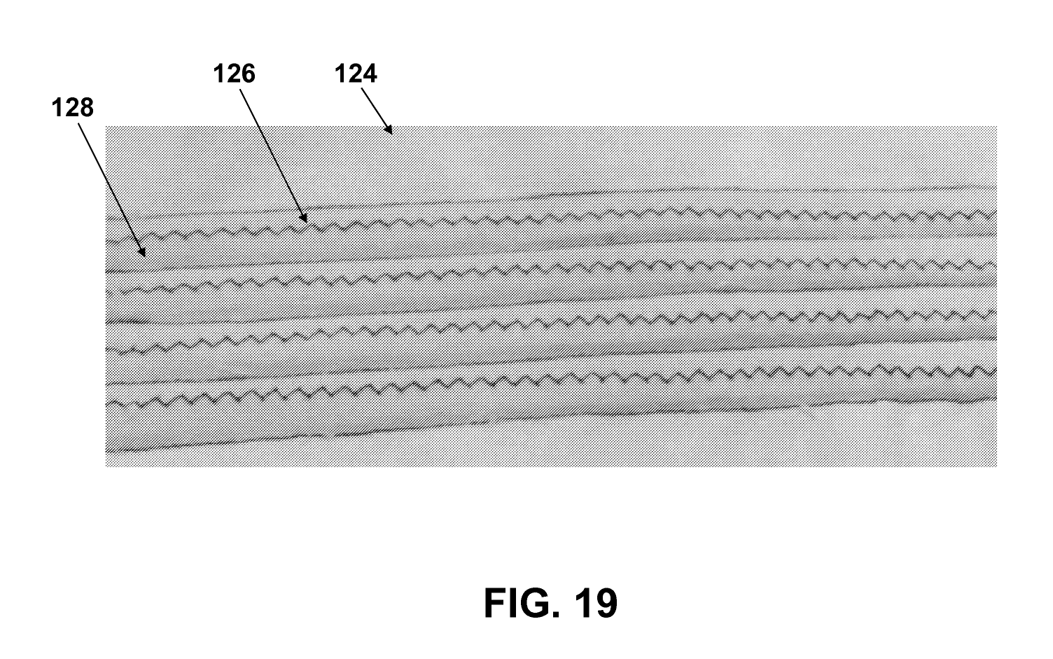

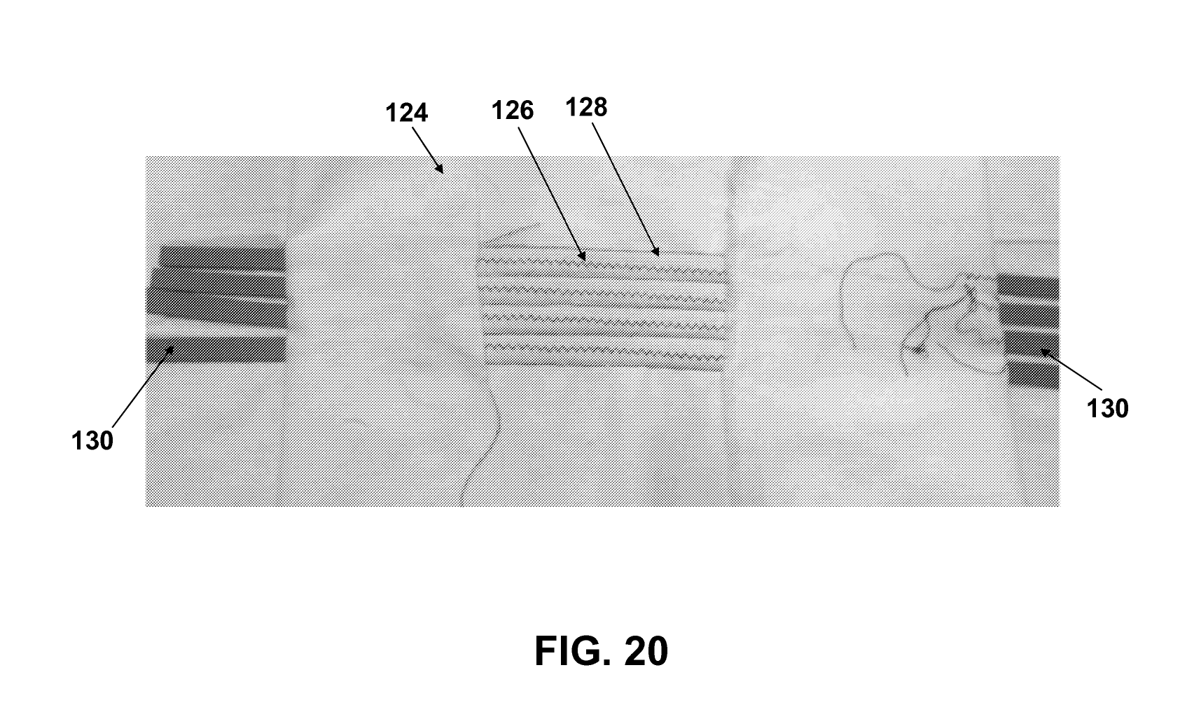

FIG. 19 shows a portion of the SMA sleeve 124 illustrating the zig-zag path 126 of the thread into the fabric of the sleeve as well as the sewn restraining tunnels 128 in order to secure the SMA in place. FIG. 20 shows the exposed rubber strips 130 inside the sleeve 124 before they were cut to length and the sleeve 124 was sewn shut. Initially, an AutoCAD drawing was created to help assist with the design and alignment of the multiple rubber strips and sewn paths. The drawings were placed underneath tissue paper and used as guides while sewing the sleeve 124 together. On the top piece of fabric, the sewn zig-zag pathways 126 served as SMA wire guides to ensure the wire aligned to its corresponding compression area. Between the two pieces of fabric, tunnels 128 for the rubber strips 130 were sewn in. These strips served to distribute the extremely focused SMA wire pressure over a larger ⅜-inch-wide area. Once the rubber strips 130 were pulled through their aligning tunnels 128, the SMA wires were threaded through the zig-zag guide 126.

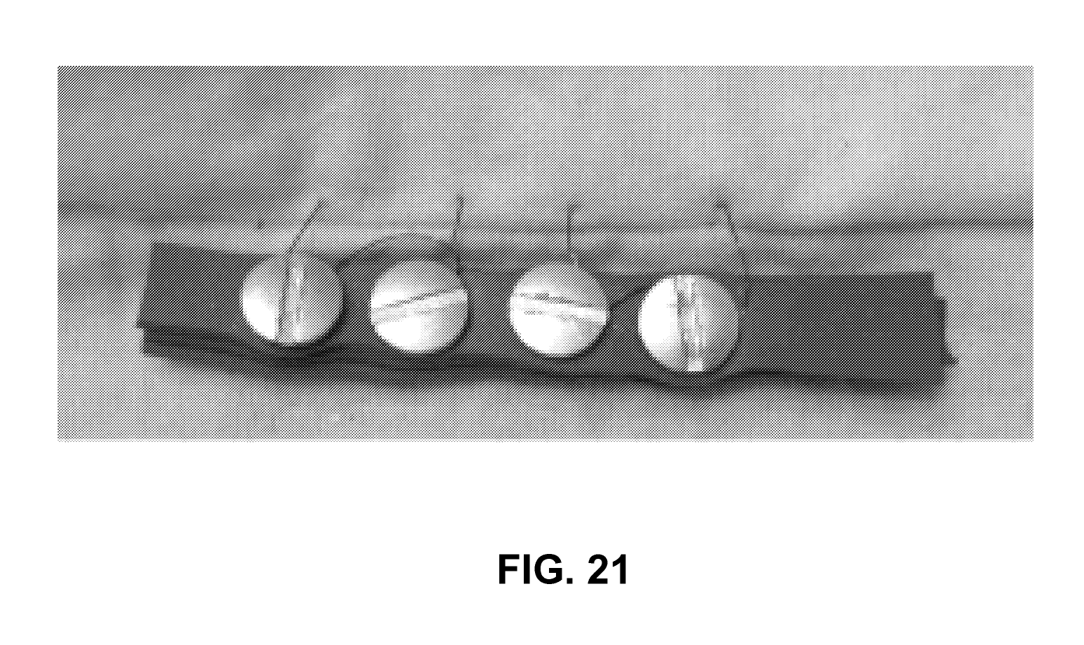

FIG. 21 shows an example internal electrical terminal with SMA wires and common return wire connected. The SMA wires were mechanically fastened down on either end via an electrical terminal. As shown in FIG. 21, one electrical terminal served as a common node and the other served as four separate inputs. The common terminal was created out of left over rubber and the ⅜″ aluminum studs.

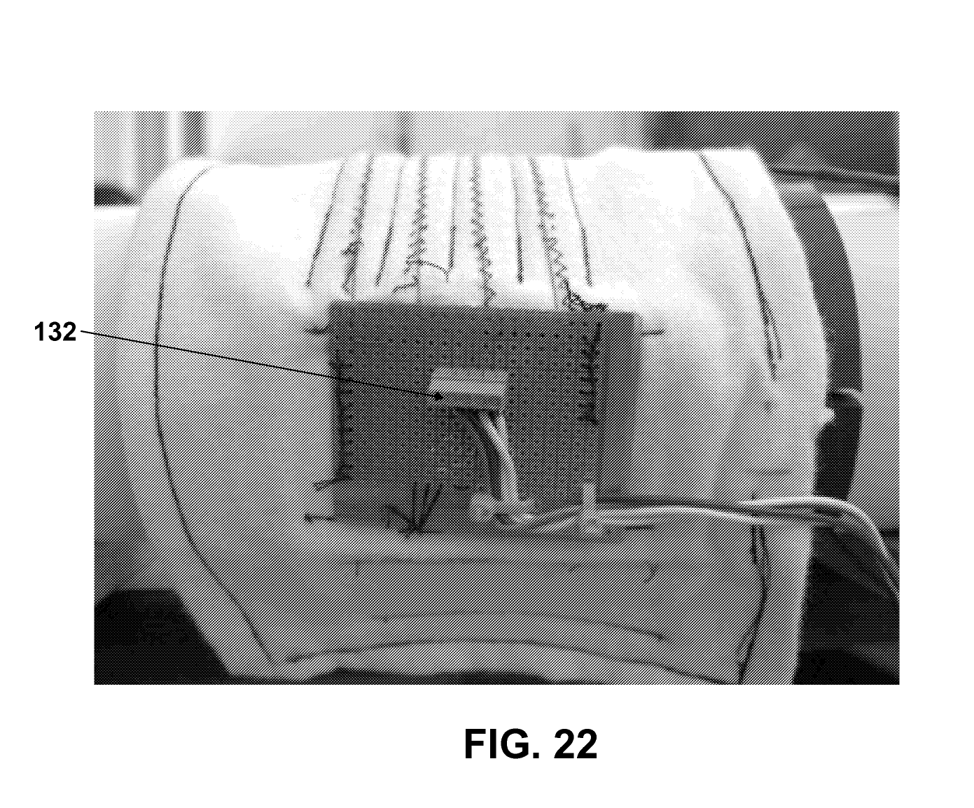

FIG. 22 shows an example external terminal 132 and power connection wires. On the remaining terminal 132, which was constructed from the solderable prototyping board, a five-input screw-terminal was soldered in and electrically connected with four colored wires that supplied power to the corresponding SMA wire.

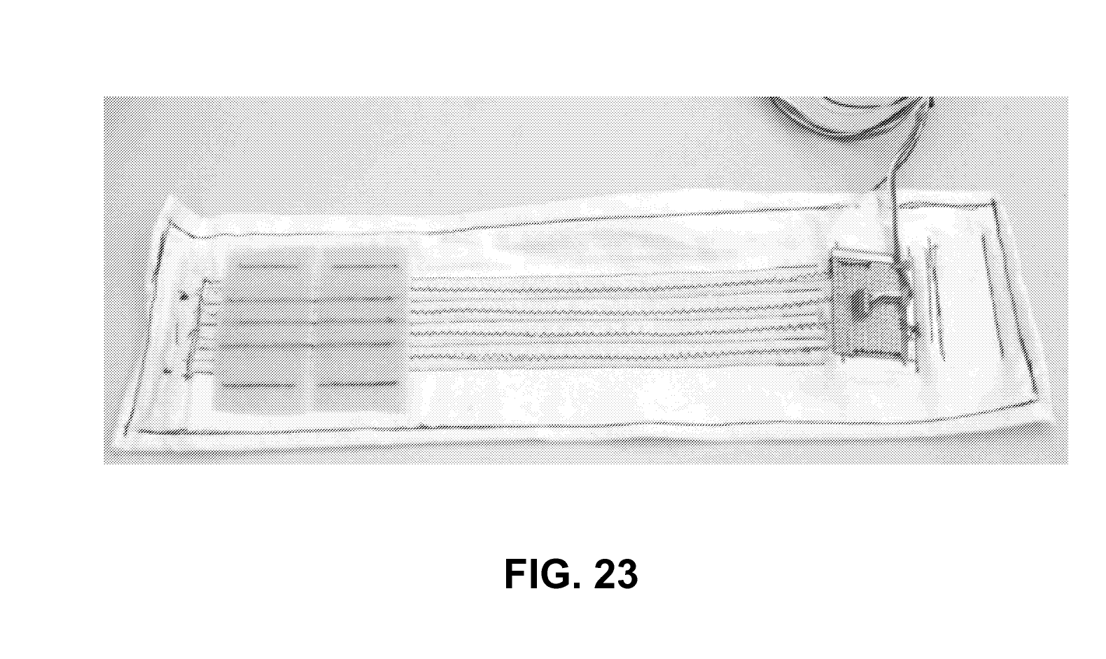

In order to mechanically secure the wires, the terminals were sewn into the sleeve as depicted in FIG. 23. The internal terminal was bounded on either side by sewing the top and bottom fabric pieces together. The external terminal (prototyping board) was sewn into the VELCRO® to ensure that it was well anchored. On either end, a four inch wide area of loop or hook VELCRO® was sewn in to the sleeve. The VELCRO® was used to fasten the sleeve.

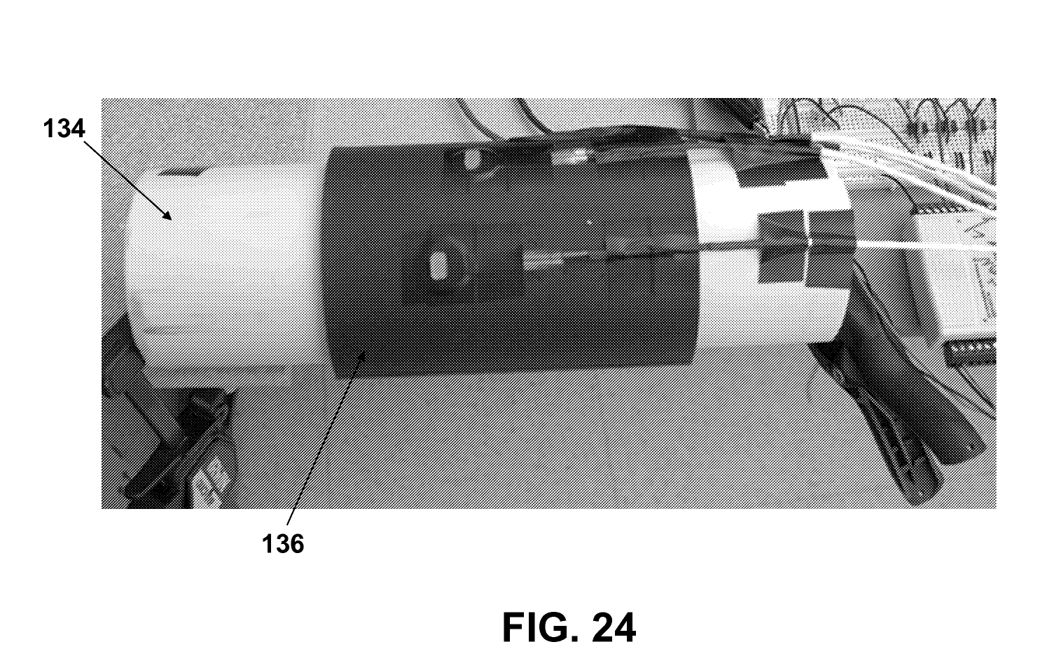

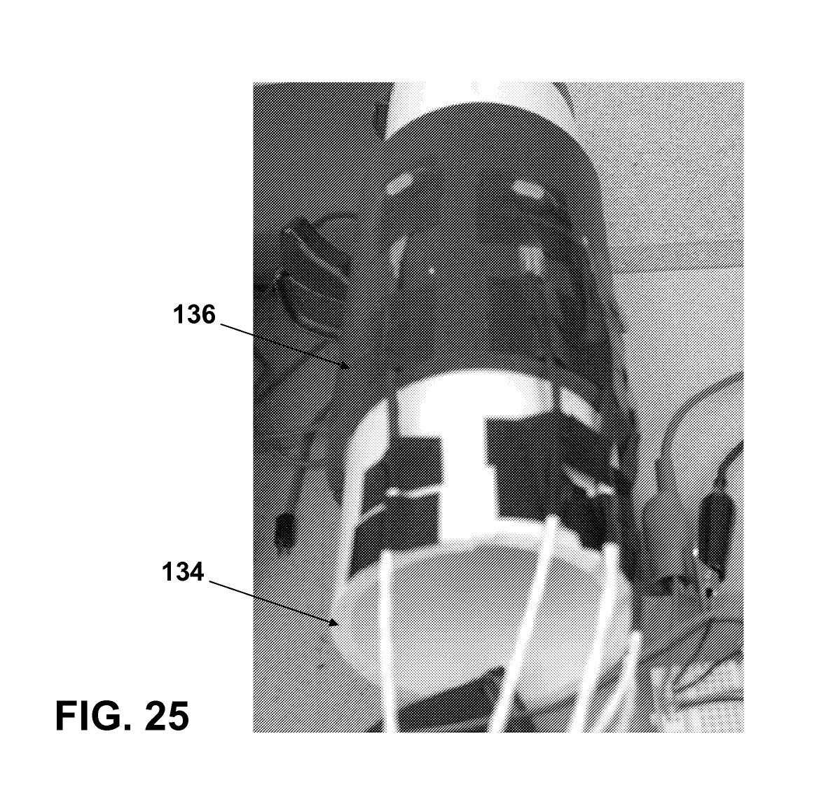

FIG. 24 and FIG. 25 show an example rig comprising a polyvinyl chloride (PVC) base 134. Also shown in FIG. 24 and FIG. 25 is a muscle-substitute foam 136 to simulate the use of the massaging sleeve on a human arm.

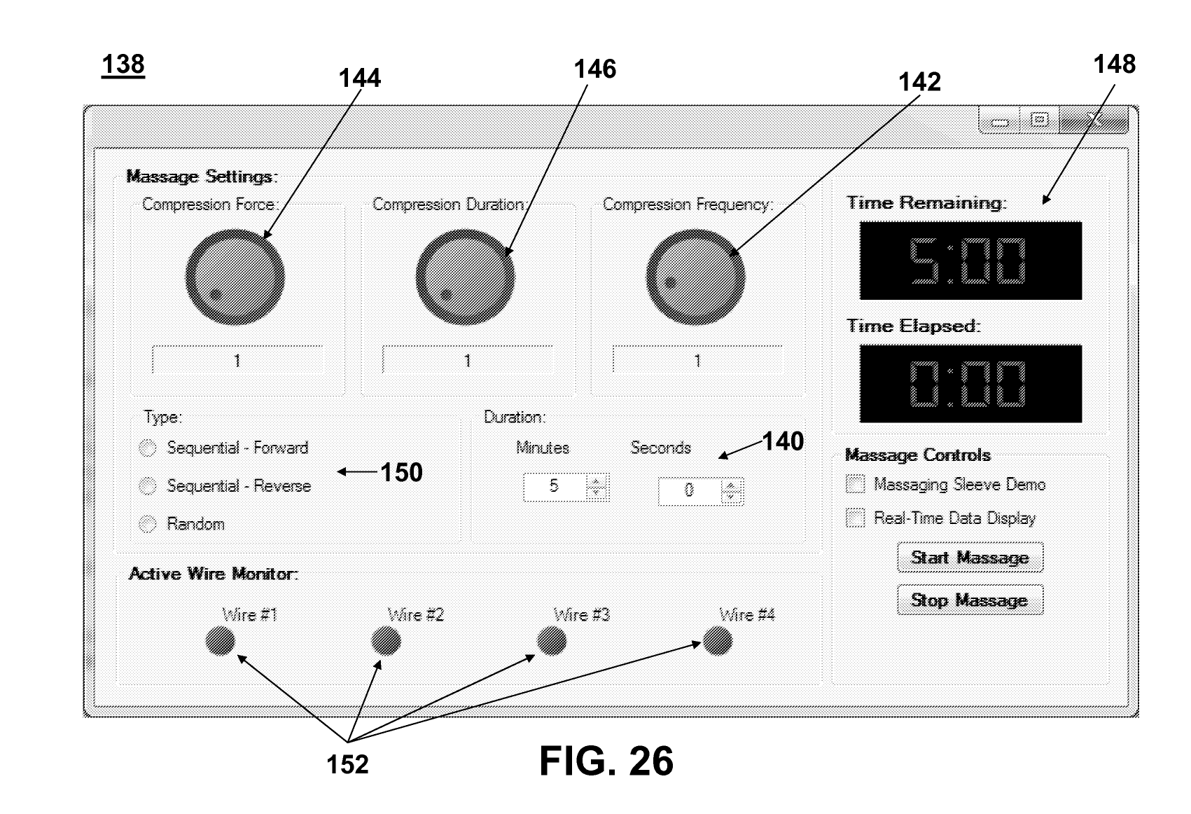

FIG. 26 depicts an example user interface 138 for controlling of the massaging sleeve. The user interface 138 may provide dynamic control of the massaging sleeve. As depicted in FIG. 26, user-controlled parameters may include run-time 140, compression frequency 142, compression force 144, and compression duration 146. Note that compression duration determines the length of time the desired compression force is maintained, and compression frequency is how often a compression occurs in a particular area. The remaining run time 148 and currently activated segment 150 also are indicated. The pattern compression type 152 determines the order of contraction of the SMA wires. In an example embodiment, a real time plotting program may be executed current with the operation of the user interface in order to show sleeve performance characteristics. Example characteristics include compression force as measured by the FSRs, SMA wire resistance, sleeve temperature, or the like, or any appropriate combination thereof.

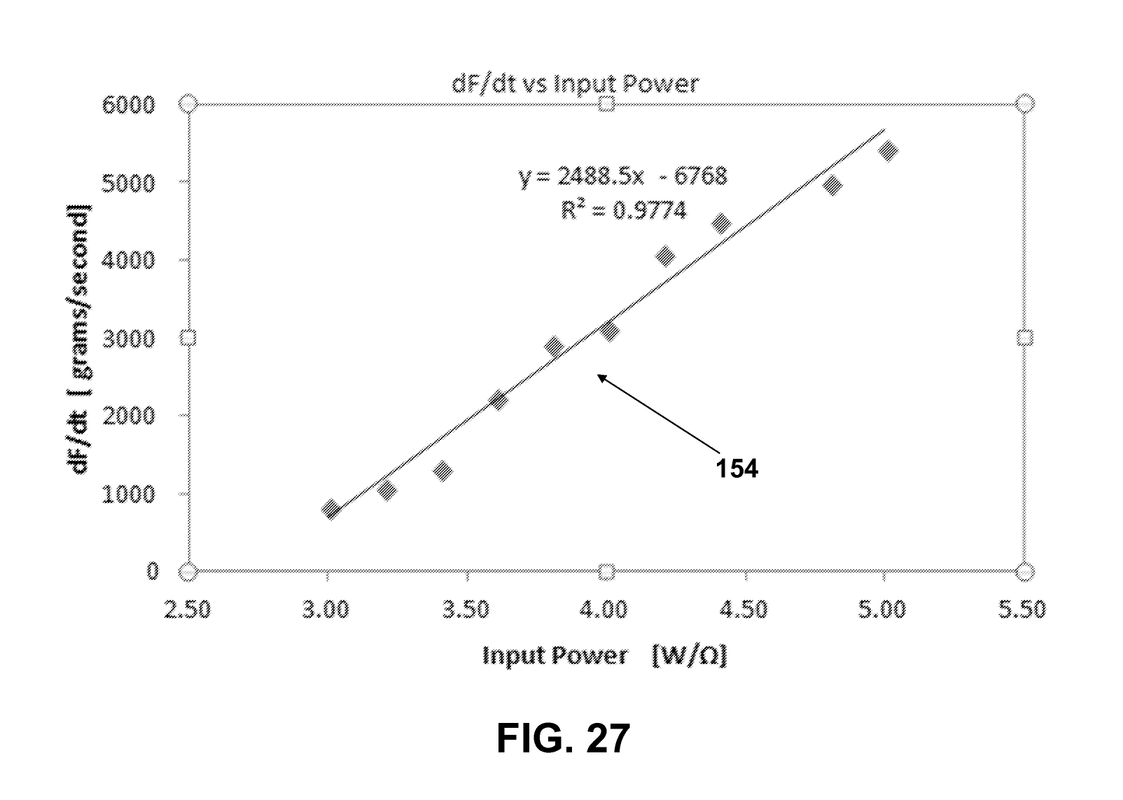

FIG. 27 is an example graph of force generated over time with respect to input power. As shown in plot 154 of FIG. 27, a the relationship between force (dF/dt in grams/second) and input power (watts/ohm) is linear. This linear relationship may be described by the following equation.

P=(2F+6.77)/2.49

where force, F_{kg}, is in kilograms and power, P, is in Watts/Ohm.

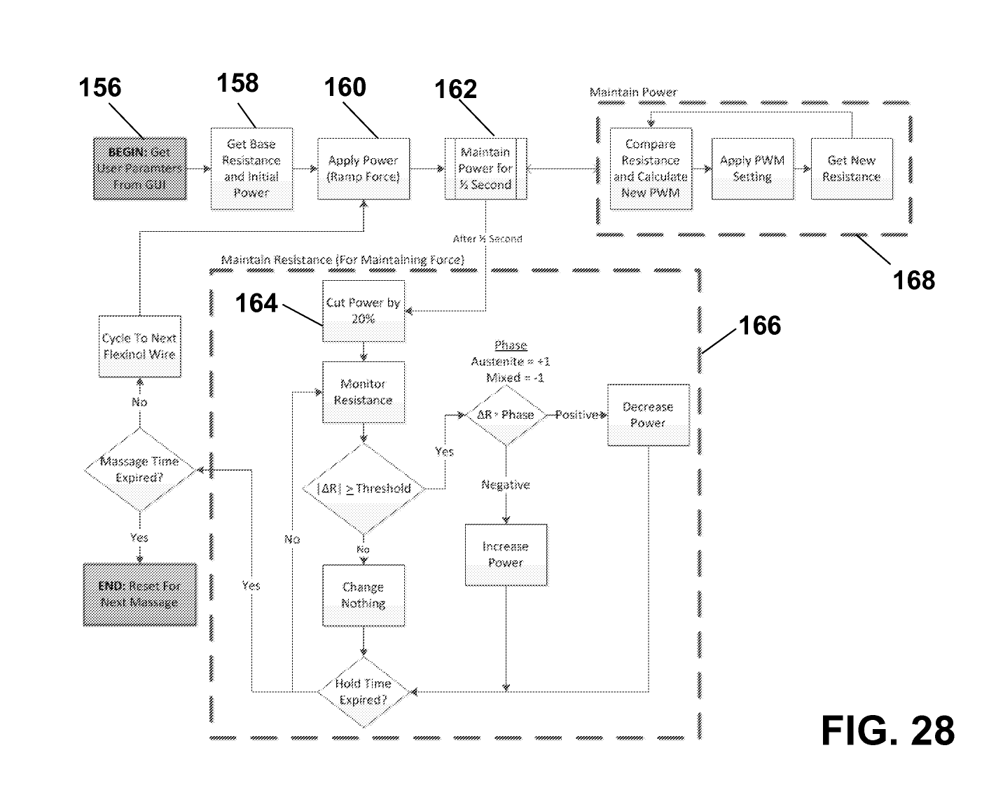

FIG. 28 is a flow diagram of an example process for controlling an SMA compression sleeve. The massage process may begin at step 156 by getting user defined parameters. The parameters may be obtained from any appropriate source, such as, for example, the user GUI. Power parameters may be calculated at step 158. Power may be applied at step 160. As power is applied, force begins to linearly ramp while power is maintained over a half second period at step 162. In an example embodiment, step 162 may apply, to a single SMA wire. In an example embodiment, step 162 may apply, to multiple SMA wires. Power may be maintained at step 168. At step 168, PWM duty cycle changes may be determined based on the difference between present input power and desired input power. After the half second period, power may be cut by 80% at step 164. In an example embodiment, resistance may be maintained throughout the process as depicted at step 166. By maintaining resistance of the SMA wire, force also may be maintained. Thus, the compression force may be held for as long as the user previously requested in the GUI. Once this hold period is over, power may be removed from the wire, and the next wire may be activated. After power is cut in step 166, using sampled resistance readings, the magnitude of the resistance difference between the current reading and value to be maintained may be checked. If below the threshold, no power adjustments may be made. If above the threshold, dependent on the wire phase and sign of difference between the current resistance and that to be maintained, either an increase or decrease in power may be made. When the hold time has elapsed, step 166 may be exited. If the pattern is to be repeated, step 160 is reached, otherwise, the program is ended.

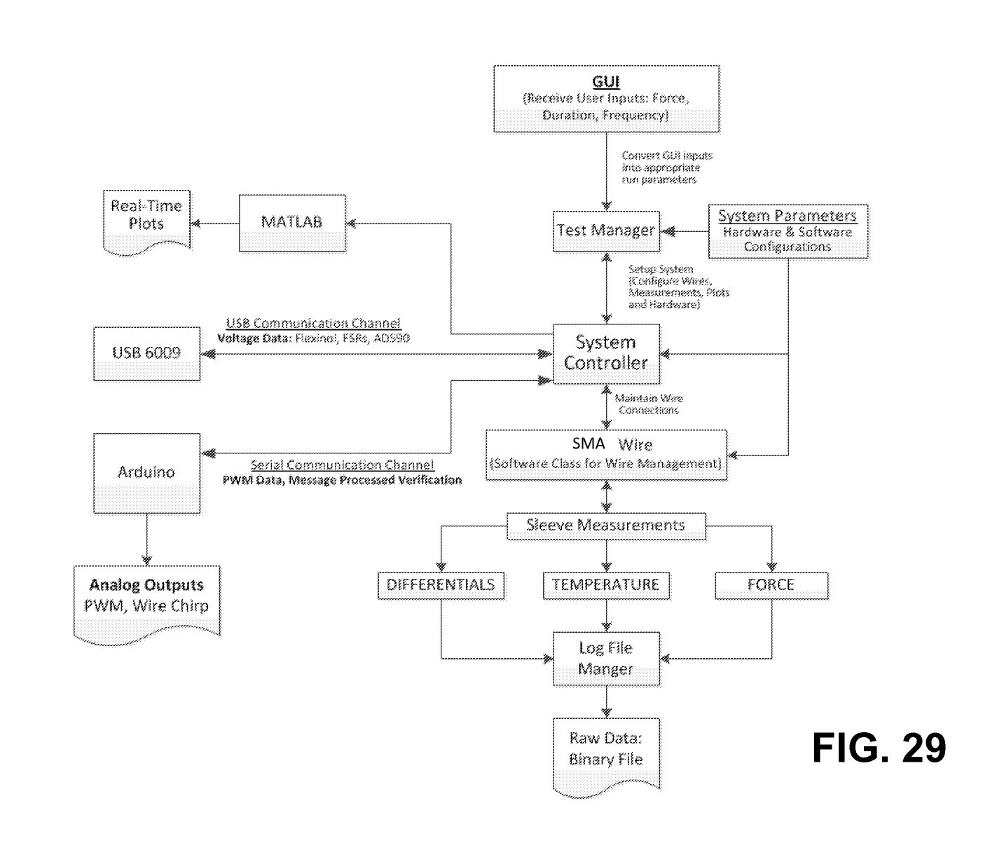

FIG. 29 is an example block diagram of a system for controlling an SMA compression sleeve. The GUI, shown in FIG. 26, may be accessed by the user and may send values to the Test Manager C# class which interfaces with the System Controller C# class. The System Controller may send data to the real-time plotting MATLAB code, receive data from the NI DAQs, send duty cycle values for the Arduino microcontroller Analog PWM signals, and interface with the C# SMA Wire class which stores data and settings for each SMA wire. The Log File Manager C# class may accumulate data and send the data to be written to binary files.

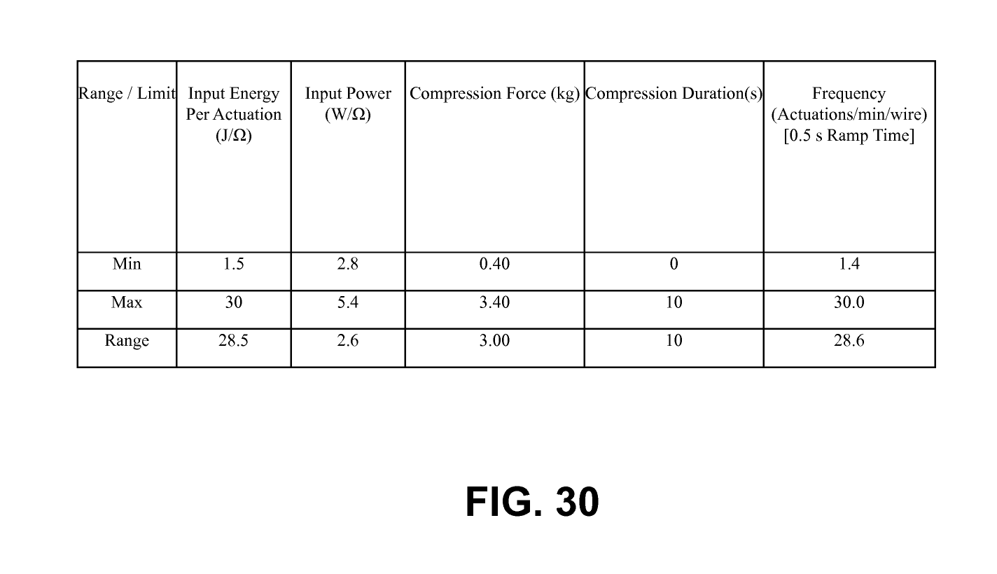

FIG. 30 shows a table of example user-available ranges of operation of the massaging sleeve. These ranges were based off of test data and FLEXINOL® specifications. Because force generation is dependent on how tight the sleeve is worn, ranges may be variable. For a tighter fit, the minimum force may be different that shown in FIG. 30, and for a looser fit, the maximum force may be different that shown in FIG. 30. Since there is a half-second force ramp period, the maximum frequency may be limited by the four half-second cycles—one for each of the four wires. For lower user-defined frequency, a non-powered wait period may be added in between actuation of each sequential wire. The maximum input energy was specified as approximately 30 J/Ω. This limit is to prevent overheating of the sleeve materials. The maximum compression duration was assigned to ten seconds. It is to be understood, however, that any appropriate time may be assigned to the maximum compression duration.

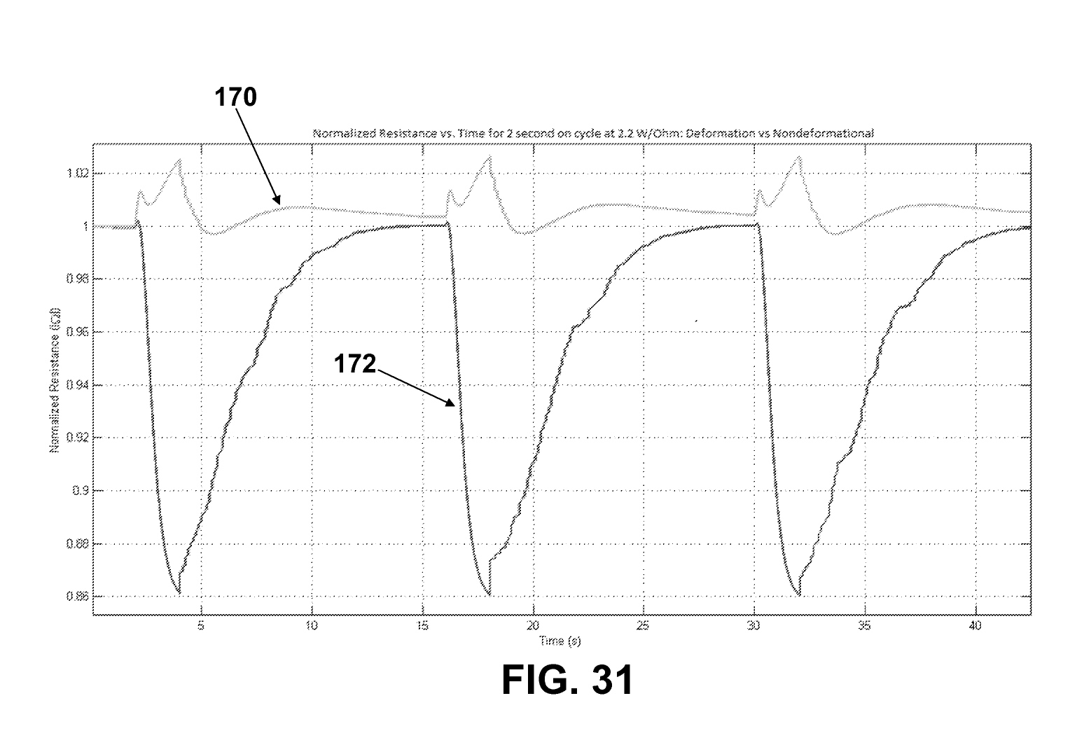

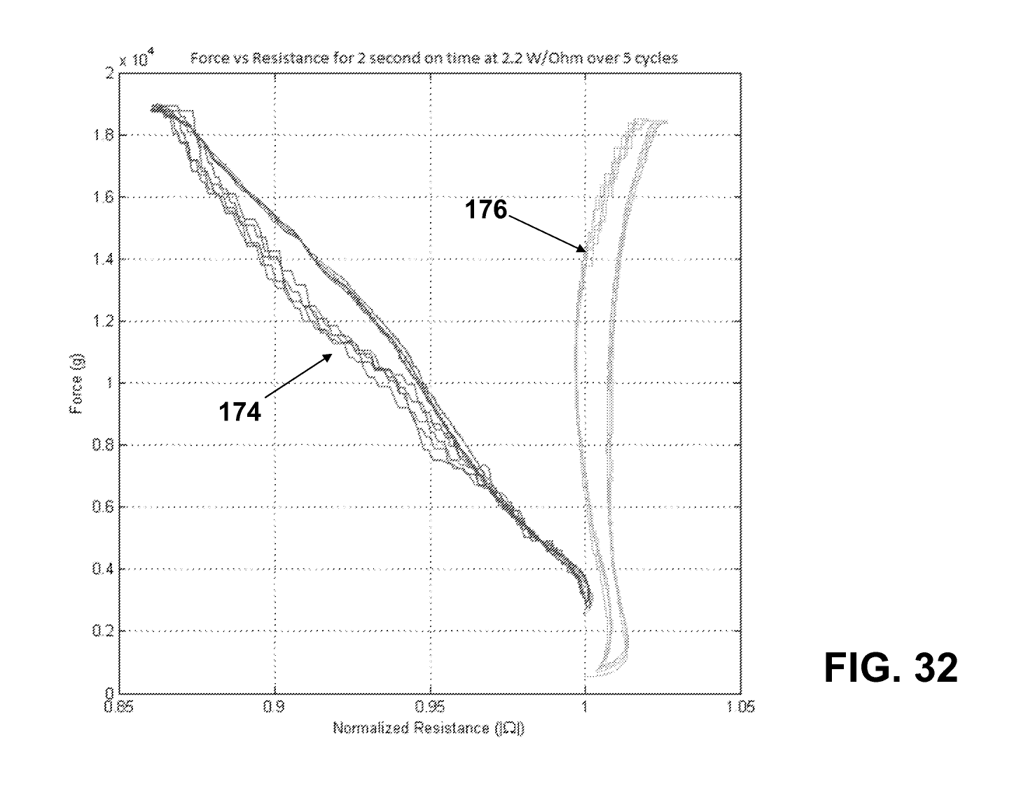

A comparison between deformation and non-deformation testing is depicted in FIG. 31 and FIG. 32. FIG. 31 illustrates example graphs of normalized resistance versus time for a two second activation period, at 2.2 W/Ω. Resistance is shown in absolute terms based on the initially measured resistance. Curve 170 is the non-deforming case and curve 172 is the deforming case. FIG. 32 illustrates example graphs of force versus normalized resistance for a two second activation period, at 2.2 W/Ω. Curve 174 is the deforming case and curve 176 is the non-deforming case. Note that the deformation case 174 shows a tilted force vs. resistance trend, however in both FIG. 31 and FIG. 32, the maximum force is the same.

The behavior of the compressible and deformable SMA material was analyzed. Differences between three characteristics of the SMA wire deformation and rigid radial compression tests were observed. First, in the deformation case, the SMA wire was allowed to contract into the deforming material (e.g., foam). In this case, the observed wire resistance dropped dramatically as the wire underwent a relatively significant length change. In the non-deforming case, the wire could not contract and its resistance behaved in an opposite manner. Referring to the resistance plots, a phase change (i.e., a minimum or maximum point) was observed to occur at different points in time. The third characteristic was that the force vs. resistance trends appeared to show the hysteric curve is tilted for the deformation case. It also was observed that for the same input power, the same peak force was reached.

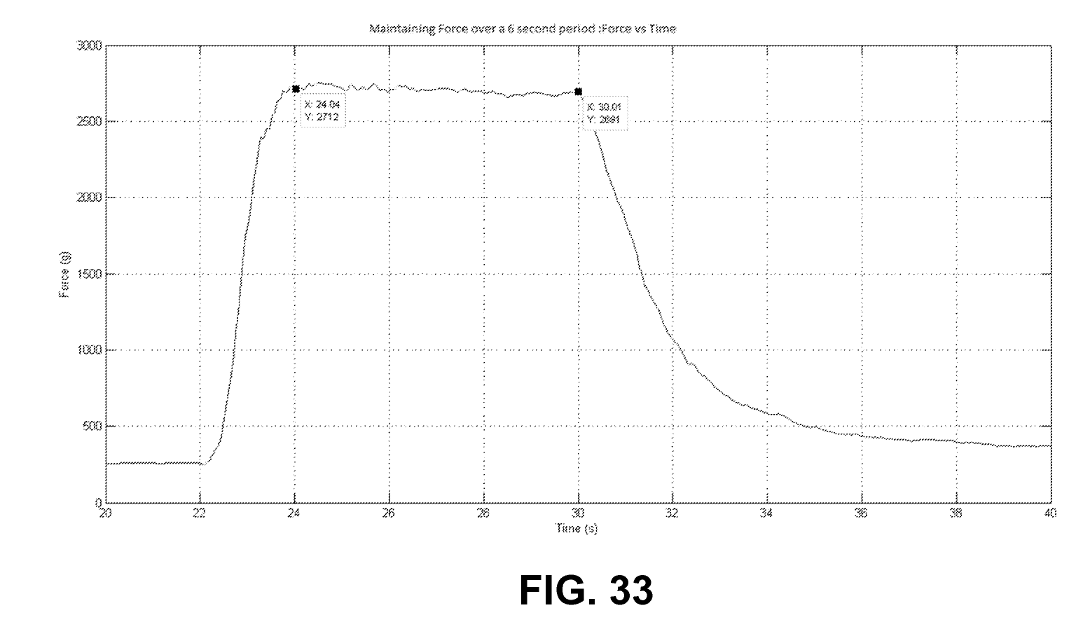

During the activation period, if powered indefinitely, the force generated via radial compression rose until it reaches a maximum force that was dictated by the SMA wire maximum recovery strength. For the 300 μm wire, this was approximately 4.2 kg of axial force. If power was removed from the wire and the wire was allowed to cool, the force decayed exponentially in conformity with typical thermodynamic cooling. If power was not completely removed, but decreased, force generation decreased, remained relatively stable, or reversed. The direction of force change depended upon how much the power to the wire was reduced. For example, when the power was dropped by 75%, the generated force decayed to a point and then reached an approximate equilibrium. At this equilibrium point the wire remained contracted with close to the same amount of compression force. While force remained constant, so did resistance. Therefore, in order to maintain the equilibrium point, and thus the compression force, resistance of the wire was not changed. An example of maintaining constant force is illustrated in FIG. 33.

FIG. 33 is an example graph depicting force vs. time wherein force was maintained over a six-second period. As shown in FIG. 33, after two seconds of activation (at overall time of 24 seconds) power was reduced by 80% and then maintained for six seconds. The starting and ending forces, which differed by only 21 grams, are 2.712 kg and 2.691 kg, respectively.

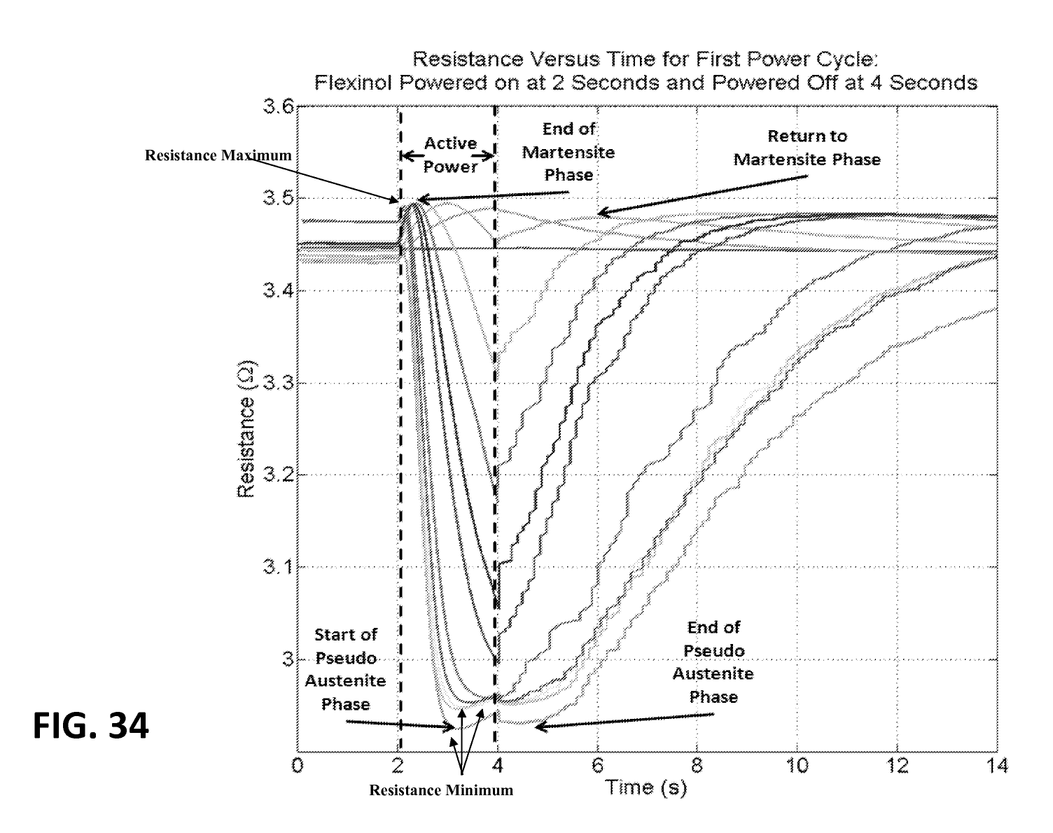

In order to analyze and characterize SMA transformation for radial compression applications of deformable bodies, six series of tests were conducted as depicted in FIG. 34 and described below. Each test was conducted with different power-on times ranging from 0.5 seconds to 3 seconds in half-second increments. For each series, the input power was incremented by 400 mW/Ω from 400 mW/Ω to 4.8 W/Ω. For each test, the SMA wire was powered and allowed to cool 5 consecutive times. Throughout all tests, the SMA wire was allowed to cool for 12 seconds after each power cycle. The wire was not touched or moved during the entire testing procedure so as not to introduce inconsistencies from changes in wire placement or incidental stress. Before testing, the constant current level of the CCS value was recorded while the control circuitry powered a dummy resistive load. During each test, local temperature, SMA wire resistance, and radial force readings were measured and recorded. After testing, all sampled data was processed to remove high frequency noise and transformed to its respective unit (° C., Ω, F_{Kg}).

In the following, resistance, temperature, and force results are reported for input power levels ranging from 0.4 W/Ω to 4.8 W/Ω, in 0.4 W/Ω steps. The power-on period for the following results is 2 seconds. Time-dependent temperature, resistance, and force trends for the remaining power-on periods are similar and therefore not discussed in the interest of brevity.

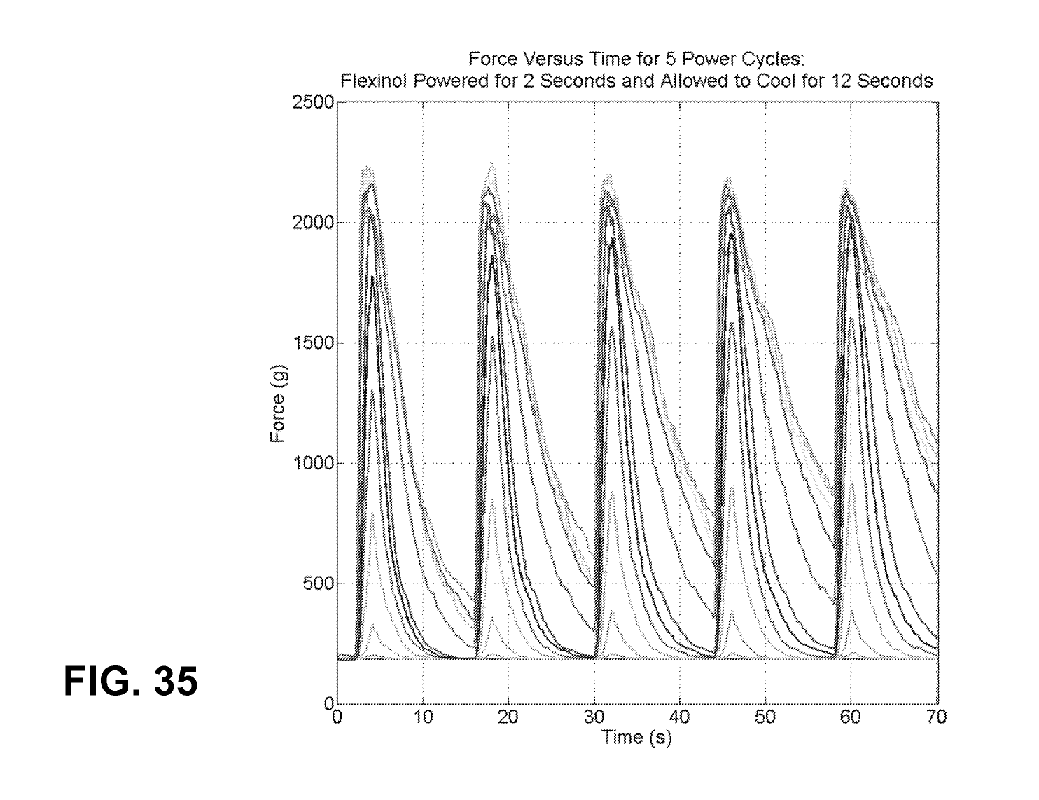

The effects on radial force exerted by the SMA wire over time were analyzed and are depicted in FIG. 35. For the 2-second period when power was supplied, the exerted force continually increased. At the highest input powers, the change in force generation dampened and stopped. Once power was removed from the wire, the force dropped off as an exponential decay. It is observed that input power was proportional to generated force.

For higher input power, the SMA wire was not given enough time to cool to relax to its initial state between subsequent power-on periods. Furthermore, over the course of the five cycles, the change in force during the cool down period decreased over subsequent cycles. These patterns may likely be the result of the foam retaining heat given off by the wire which is then radiated back into the SMA wire.

The changes in resistance of the SMA wire versus time were analyzed. Resistance curves for a single power and cool down cycle indicated that the first maximum and minimum in a cycle on the resistance corresponded to the beginning and the end of the M/Pseudo-A phase transformation, respectively. For the 0.4 W/Ω input power level, the SMA wire had a negligible response. Input powers between 0.8 W/Ω and 2.8 W/Ω indicated a partial phase transformation.

Input powers of 3.2 W/Ω to 4.8 W/Ω indicated a full transformation into the Pseudo-Austenite state. Once power was turned off, the SMA wire underwent the Pseudo-A/M transformation. Once the wire began to cool, the resistance began to settle towards its full martensite value. For some results, the lack of a maximum in the non-powered state indicated that the wire did not cool sufficiently to fully return to the complete martensite state. In these cases, despite lacking of a full phase-cycle, the resistance response in subsequent cycles behaved as a wire would that starts in the Mixed phase.

Changes in the local temperature versus time were analyzed. The results indicate the expected trend of faster rates of change and larger maximum temperatures for higher input powers. The initial temperature for each test was kept within a range of 22-28® C. The increase in a wire's starting temperature was the result of inadequate cooling time between individual tests. While not ideal, a 6® C. initial temperature fluctuation played only a minor role in describing the force generation behavior of SMA wire for radial actuation.

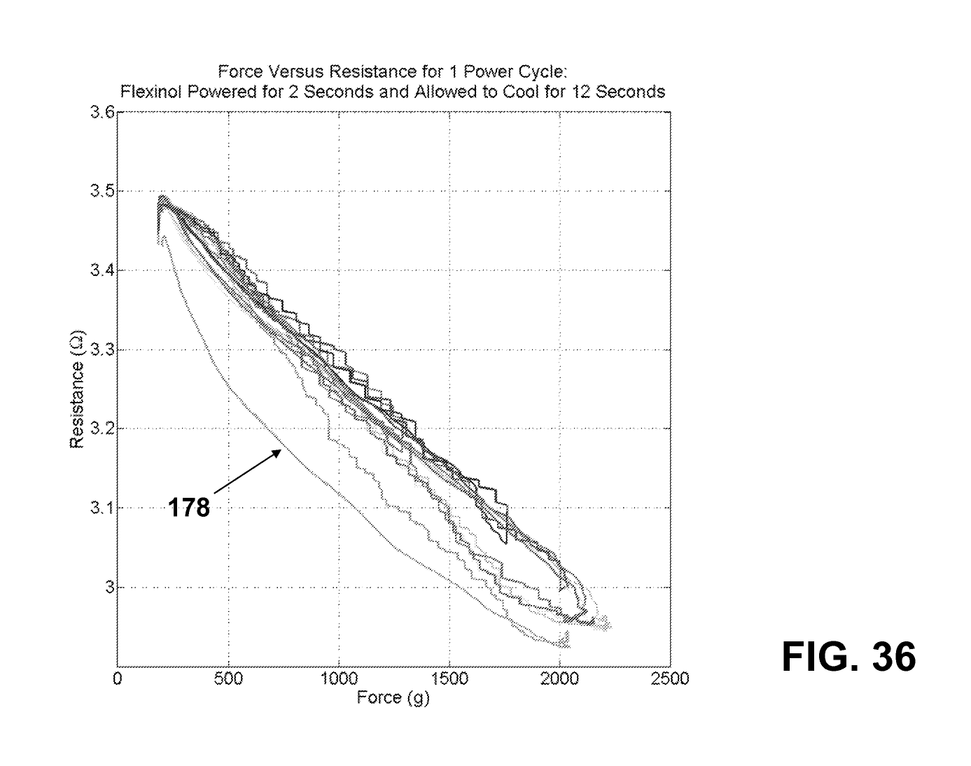

FIG. 36 is an example graph of resistance of the SMA wire vs. radial force exerted by the SMA wire. Resistance is presented on the vertical axis to better indicate wire phase status. Note that for higher input power, the overall curve tends to have a lower offset. FIG. 36 shows the relationship between force and resistance over the first cycle of each test where the power-on period is 2 seconds. The quantized portions of the curves is a result of sampling the wire at a low frequency after the active power period. Over the set of curves depicted in FIG. 36, a negative correlation between resistance and force was observed. Except for the bottom most curve 178 (4.0 W/Ω), most of the curves follow close to the same path. The bottom curve 178 may be the result of a sudden temperature shift in the testing environment.

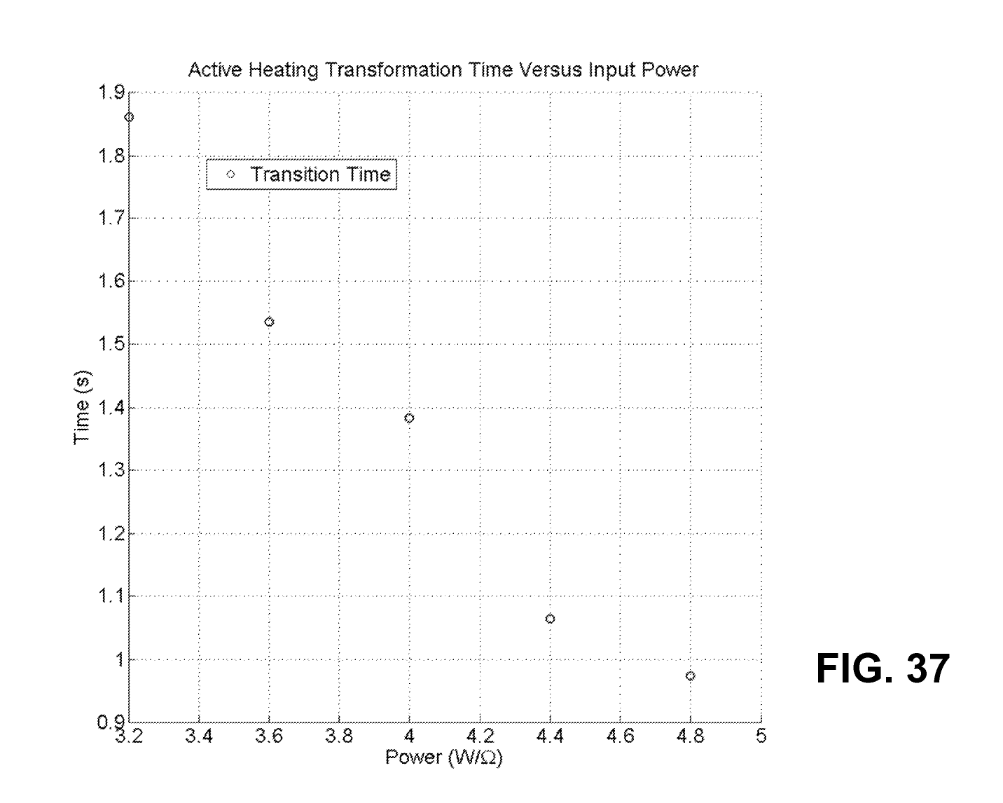

FIG. 37 is an example plot depicting transformation (transition) times vs. power. As shown in FIG. 37, transformation time decreased as input power increased. The time during the mixed phase illustrated how much faster phase change of the SMA wire can be induced. Multiple data points for different power-on times were averaged together to yield the data represented in FIG. 37. Note that the five highest power tests for the 2.0 s, 2.5 s and 3.0 second test series achieved full M/Pseudo-A transformation. A negative correlation between input power and transformation time is apparent.

In addition to looking at transformation time, the rate of change of wire resistance with respect to time provided information about the dynamic response of the actuating wires. This was given as the change in resistance divided by the length of time in a corresponding phase. The time in each phase was dictated as follows: Martensite) Power-on timer mark until first maximum, Mixed) First maximum to first minimum, Pseudo-Austenite) First minimum until power-off mark.

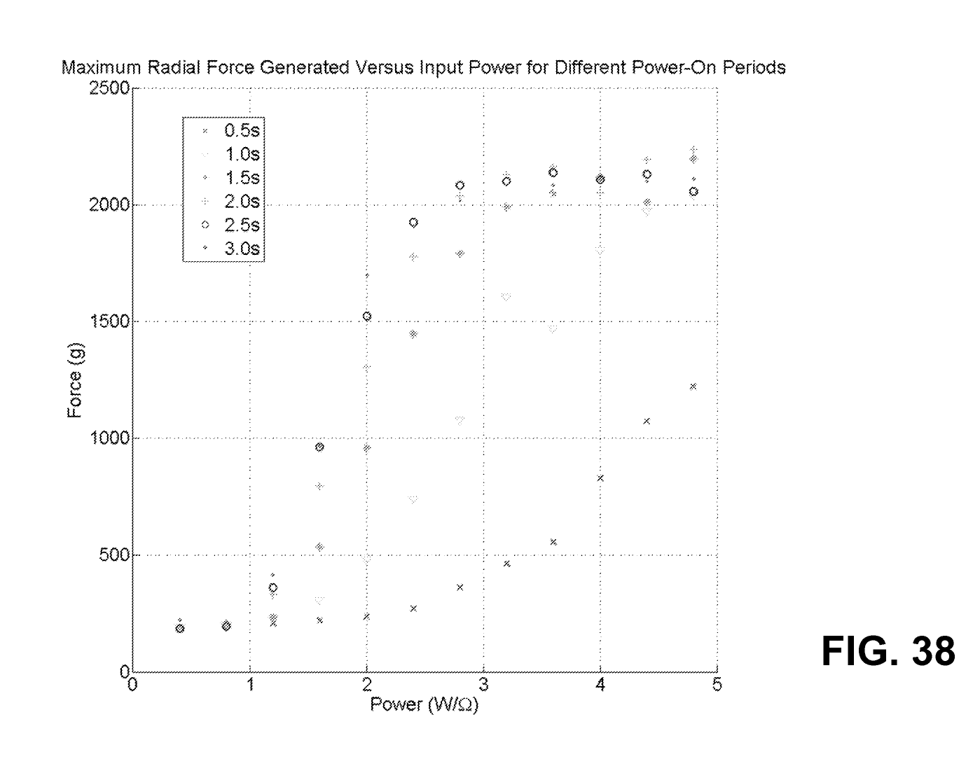

FIG. 38 is an example plot depicting maximum force vs. power for different power-on times. The maximum forces observed were approximately 2.1 kg. The SMA wire was not capable of completely compressing the foam layer; the required change in wire length (or outer rig circumference) exceeded the wire's maximum length change. Therefore, when the wire was maximally contracted, the maximum force achieved was not a function of the SMA wire's maximum operating force. Rather, it is a function of the rigidity (or softness) of the material being deformed.

Several observations were made during the foregoing experiments and analysis regarding SMA transformation for radial compression applications of deformable bodies. There was a positive correlation between input power for a given power-on period and maximum generated force. The rate change of resistance was significantly higher in the Pseudo-A-phase than M-phase (at higher power values). This differs from the linear actuation case. There was a positive correlation between input power and rate change of resistance in the Pseudo-A-phase. An increase in power supplied to the wire correlated to a shorter transition time.

In various example embodiments, a device comprising a shape-memory-alloy may be in the form of a sleeve, a wrap, a cuff, or the like. For example, the device may be in the form of a sleeve that may be placed around a limb of a person or animal. The shape-memory-alloy may be in the form of a wire and/or tape. The shape-memory-alloy may be provided constant electrical current. The shape-memory-alloy may be provided constant electrical voltage. The shape-memory-alloy may be provided constant electrical power. The shape-memory-alloy may be provided pulsed electrical current. The shape-memory-alloy may be provided pulsed electrical voltage. The shape-memory-alloy may be provided pulsed electrical power. The shape-memory-alloy may be provided predetermined electrical current profile. The shape-memory-alloy may be provided predetermined electrical voltage profile. The shape-memory-alloy may be provided predetermined electrical power profile. The shape-memory-alloy may be provided any appropriate combination of electrical current, voltage, and power as described above. In an example embodiment, the shape-memory-alloy may be provided a constant electrical current and a pulsed electrical voltage. Values of the constant electrical current and a pulsed electrical voltage may be such that a predetermined value of electrical power is maintained. In an example embodiment, the device may provide concurrent heat compression. In an example embodiment, a specific configuration of the shape-memory-alloy may be used to facilitate manufacturing the shape-memory-alloy. For example, dimensions of a person's limb (e.g., ankle, calf, arm, etc.) and amount of compression needed to treat the person may be determined Additionally, an amount of heat needed to treat a person may be determined. The determined dimensions, the amount of compression, and/or the amount of heat may be provide to a manufacturer, or the like, of shape-memory-alloys in order to produce a shape-memory-alloy that is tailored to the person's needs.

Radial compression utilizing a shape memory alloy may be effectuated by a device, processor, or the like. Radial compression utilizing a shape memory alloy may be controlled by processor, or the like, to apply a compressive force. For example, a processor may be coupled to a memory that that comprises executable instructions, that when executed by the processor cause the processor to effectuate operations for effectuating radial compression utilizing a shape memory alloy. The underlying concepts may be applied to any computing device, processor, or system capable of controlling the device. Certain aspects or portions thereof, may take the form of program code (e.g., instructions) embodied in computer-readable storage media having a tangible physical structure. Examples of computer-readable storage media include floppy diskettes, CD-ROMs, DVDs, hard drives, or any other tangible machine-readable storage medium (computer-readable storage medium) having a tangible physical structure. Thus, a computer-readable storage medium is not a transient signal per se. A computer-readable storage medium is not a propagating signal per se. A computer-readable storage medium is an article of manufacture. When the program code is loaded into and executed by a machine, such as a computer or processor, the machine becomes an apparatus for controlling the device.

While radial compression utilizing a shape memory alloy has been described in connection with the various embodiments of the various figures, it is to be understood that other similar embodiments may be used or modifications and additions may be made to the described embodiments of radial compression utilizing a shape memory alloy without deviating therefrom. For example, one skilled in the art will recognize that embodiments and application of radial compression utilizing a shape memory alloy as described in the instant application may apply to any appropriate environment, and may be applied to any number of devices. Therefore, radial compression utilizing a shape memory alloy as described herein should not be limited to any single embodiment, but rather should be construed in breadth and scope in accordance with the appended claims.

What is claimed:

1. An apparatus comprising:
a first component comprising a shape-memory alloy, the component providing a compressive force responsive to applied electrical energy; and
a second component that provides electrical energy to the first component.

2. The apparatus of claim 1, wherein the first component provides heat responsive to the applied electrical energy.

3. The apparatus of claim 1, wherein the shape-memory-alloy comprises nickel titanium.

4. The apparatus of claim 1, wherein the first component is shaped as a wire.

5. The apparatus of claim 1, wherein the first component is shaped as a coil.

6. The apparatus of claim 1, wherein:
the first component is shaped as a coil; and
the compressive force is in a direction toward a center of the coil.

7. The apparatus of claim 1, wherein electrical energy is provided to the first component in accordance with a predetermined duty cycle.

8. The apparatus of claim 1, wherein:
the first component comprises a plurality wires;
the plurality of wires are configured to form a coil;
each wire of the plurality of wires comprises a shape-memory alloy; and
each wire of the plurality of wires is individually controlled by the second component.

9. An apparatus comprising:
a processor; and
memory coupled to the processor, the memory comprising executable instructions that when executed by the processor cause the processor to effectuate operations comprising:
providing, via a shape-memory alloy, a radially inward compressive force; and
providing, concurrent with providing the radially inward compressive force, via the shape-memory alloy, thermal energy.

10. The apparatus of claim 9, wherein the shape-memory-alloy comprises nickel titanium.

11. The apparatus of claim 9, wherein the radially inward compressive force and the thermal energy are provided via a wire comprising the shape-memory alloy.

12. The apparatus of claim 11, wherein:
the wire is configured as a coil; and
the compressive force is in a direction toward a center of the coil.

13. The apparatus of claim 9, wherein:
the compressive force is provided responsive to electrical energy that is provided in accordance with a duty cycle.

14. The apparatus of claim 9, wherein:
the radially inward compressive force and the thermal energy are provided via a plurality of wires;
the plurality of wires is configured to form a coil;
each wire of the plurality of wires comprises a shape-memory alloy; and
each wire of the plurality of wires is individually controlled to provide the radially inward compressive force and the thermal energy.

15. A computer readable storage medium comprising executable instructions that when executed by a processor cause the processor to effectuate operations comprising:
providing, via a shape-memory alloy, a radially inward compressive force; and
providing, concurrent with providing the radially inward compressive force, via the shape-memory alloy, thermal energy.

16. The computer readable storage medium of claim 15, wherein the shape-memory-alloy comprises nickel titanium.

17. The computer readable storage medium of claim 15, wherein the radially inward compressive force and the thermal energy are provided via a wire comprising the shape-memory alloy.

18. The computer readable storage medium of claim 17, wherein:
the wire is configured as a coil; and
the compressive force is in a direction toward a center of the coil.

19. The computer readable storage medium of claim 15, wherein:
the compressive force is provided responsive to electrical energy that is provided in accordance with a duty cycle.

20. The computer readable storage medium of claim 15, wherein:
the radially inward compressive force and the thermal energy are provided via a plurality of wires;
the plurality of wires is configured to form a coil;
each wire of the plurality of wires comprises a shape-memory alloy; and
each wire of the plurality of wires is individually controlled to provide the radially inward compressive force and the thermal energy.
